(19) United States
(12) Patent Application Publication
(10) Pub. No.: US 2003/0108906 A1
(43) Pub. Date: Jun. 12, 2003

(54) Identification and use of molecules implicated in pain

(76) Inventors: Robert Alan Brooksbank, Cambridge (GB); Alistair Kerr Dixon, Cambridge (GB); Kevin Lee, Cambridge (GB); Robert Denham Pinnock, Ann Arbor, MI (US)

Correspondence Address: Mehdi Ganjeizadeh, Ph.D., Warner-Lambert Company, 2800 Plymouth Road, Ann Arbor, MI, 48105, US

(21) Appl. No.: 10/205,342

(22) Filed: Jul. 24, 2002

(30) Foreign Application Priority Data
Feb. 7, 2002 (GB) 0202892.6
Jul. 27, 2001 (GB) 0118354.0

(51) Int. Cl. A01K 67/033; G01N 33/00; C12Q 1/68; G01N 33/53; G01N 33/567

(52) U.S. Cl. 435/6; 435/7.2; 800/3; 800/13

(57) ABSTRACT

The invention relates to the use of:

Use of:

(a) an isolated gene sequence that is up-regulated in the spinal cord of a mammal in response to mechanistically distinct first and second models of neuropathic or central sensitization pain;

(b) an isolated gene sequence comprising a nucleic acid sequence of Table 1;

(c) an isolated gene sequence having at least 80% sequence identity with a nucleic acid sequence of Table 1;

(d) an isolated nucleic acid sequence that is hybridizable to any of the gene sequences according to (a), (b) or (c) under stringent hybridisation conditions;

(e) a recombinant vector comprising a gene sequence or nucleic acid sequence according to any one of (a) to (d);

(f) a host cell comprising the vector according to (e);

(g) a non-human animal having in its genome an introduced gene sequence or nucleic acid sequence or a removed or down-regulated gene sequence or nucleic acid sequence according to any one of (a) to (d);

(h) an isolated polypeptide comprising an amino acid sequence at least 90% identical to an amino acid sequence encoded by a nucleotide sequence according to any one of (a) to (d), or a polypeptide variant thereof with sequential amino acid deletions from the C terminus and/or the N-terminus;

(i) an isolated polypeptide encoded by a nucleotide sequence according to any one of (a) to (d); or

(j) an isolated antibody that binds specifically to a polypeptide according to (h) or (i);

in the screening of compounds for the treatment of pain, or for the diagnosis of pain.

The invention also relates to the use of naturally occurring compounds such as peptide ligands of the expression products of the above gene sequences and their associated signal transduction pathways for use in the treatment of pain.

This application claims the priority of United Kingdom application NO. 0118354.0, filed Jul. 27, 2001, and United Kingdom application NO. 0202892.6, filed Feb. 7, 2002; the entire contents of which applications are hereby incorporated by reference herein.

### FIELD OF THE INVENTION

The present invention relates to nucleic acids, their expression products and pathways involved in pain, and their use in screening for molecules that can alleviate pain. The invention further relates to methods for the assay and diagnosis of pain in patients.

### BACKGROUND OF THE INVENTION

Pain is currently classified into four general types. Post-operative acute pain can be successfully treated with existing pain medications of e.g. the opioid and non-steroidal anti-inflammatory (NSAID) types, and is usually short-term and self-limiting. A second type of pain, present e.g. in cancer and arthritis, is also responsive to medication initially with a NSAID and in its later stages with opioids. Neuropathic pain arises from damage to the central or peripheral nerve systems, and is more effectively treated with antidepressants or anticonvulsants. A fourth type of pain called central sensitization results from changes in the central nervous system as a result of chronic pain, these changes often being irreversible and difficult to treat. Nerve pain from shingles or diabetes falls into this and the neuropathic category. Changes occur where pain is at first poorly controlled and gradually progress to the point where a person is sensitive to stimuli which would not normally cause pain, for example a light touch. People with pain of this kind often describe a widening of the pain area to include areas which had originally not been injured or which were thought not to be involved in pain. This classification is, however based on clinical symptoms rather than on the underlying pain mechanisms.

Opiates such as morphine belong to a traditional class of pain-relieving compounds that are now recognized as binding to opiate receptors. Naturally occurring polypeptides have also been found to have opiate-like effects on the central nervous system, and these include β-endorphin, met-enkephalin and leu-enkephalin.

Salicin was isolated at the beginning of the 19th century, and from that discovery a number of NSAIDs such as aspirin, paracetamol, ibuprofen, flurbiprofen and naproxen were developed. NSAIDs are by far the most widely used pain-relieving compounds, but can exhibit side effects, in particular irritation of the GI tract that can lead to the formation of ulcers, gastrointestinal bleeding and anemia.

Interest in the neurobiology of pain is developing: see a colloquium sponsored by the US National Academy of Sciences in December 1998 concerning the neurobiology of pain and reviewed in The Scientist 13[1], 12, 1999. Many pain mechanisms were discussed including the role of the capsaicin receptors in pain, (M. J. Caterina et al., Nature, 389, 816-824, 1997). Large dosages of capsaicin were reported to disable that receptor, (W. R. Robbins et al., Anesthesia and Analgesia, 86, 579-583, 1998). Additionally, a tetrodotoxin-resistant sodium channel found in small diameter pain-sensing neurons (PN3) was discussed (A. N. Akopian et al., Nature, 379, 257-262, 1986) and L. Sangameswaran et al., Journal of Biological Chemistry, 271, 953-956, 1996). Its involvement in transmission and sensitization to pain signals has been reported, (S. D. Novakovic et al., Journal of Neuroscience, 18, 2174-2187, 1998). A further tetrodotoxin-resistant sodium channel has been reported (S. Tate et al., Nature Neuroscience, 1, 653-655 1998).

Second messenger systems have also been shown to be important since knockout-mice lacking protein kinase C (PKC) γ were reported to respond to acute pain e.g. from a hot surface, but not to respond to neuropathic pain when their spinal nerves are injured (Malmberg et al., Science, 278, 279-283 (1997).

Present methods for identifying novel compounds that relieve pain of one or more of the types indicated above suffer from the defect that they are dependent either on the relatively limited number of receptors known to be involved in pain or on the empirical identification of new receptors which is an uncertain process. In relation to known receptors, for example the opioid receptor, research directed to improved compounds offers the possibility of screening compounds that have a better therapeutic ratio and fewer side effects. This does not lead naturally to compounds for different pain receptors that have new modes of action and new and qualitatively different benefits. Even when newly identified additional receptors are taken into account, known receptors revolve around tens of gene products. However, there are between 30,000 and 40,000 genes in the genome of an animal and more of them are concerned with nervous system function than with peripheral function. We therefore concluded that a large number of receptors and pathways are important to the transduction of pain, but up to now have remained unknown.

### SUMMARY OF THE INVENTION

It is an object of the invention to provide sequences of genetic material for which no role in pain has previously been disclosed, and which are useful, for example, in:

identifying metabolic pathways for the transduction of pain

identifying from said metabolic pathways compounds having utility in the diagnosis or treatment of pain

producing proteins and polypeptides with a role in the transduction of pain;

producing genetically modified non-human animals that are useful in the screening of compounds having utitlity in the treatment or diagnosis of pain.

Identifying ligand molecules for receptors involved in said metabolic pathways and having utility in the treatment of pain.

It is yet a further object of the invention to provide research tools, for example non-human animals and microorganisms, that can be used in screening compounds for pharmacological activity, especially pain-reducing activity.

The present invention is based on sequences that are up-regulated in two models of chronic pain, namely streptozocin-induced diabetes and chronic constrictive injury (CCI) to a nerve leading to the spine, for example the sciatic nerve.

In one aspect, the invention relates to the use in the screening of compounds that are effective in the treatment of pain, or in the diagnosis of pain, of:

(a) an isolated gene sequence that is up-regulated in the spinal cord of a mamal in response to first and second models of pain, for example in response to streptozocin-induced diabetes and in response to a chronic constrictive injury to a nerve leading into the spine;

(b) an isolated gene sequence having at least 80% sequence identity with any of the the nucleic acid sequences of the accompanying Table I in the specification, preferably 85% sequence identity, more preferably 90%, increasingly preferably 95%, most preferably 99%.

(c) an isolated nucleic acid comprising a sequence that is hybridizable to any of the gene sequences according to (a) or (b) under stringent hybridisation conditions;

(d) a recombinant vector comprising any one of the gene sequences according to (a) to (c);

(e) a host cell containing the vector according to (d);

(f) a non-human animal, for use in the screening of compounds that are effective in the treatment of pain, or in the diagnosis of pain, having in its genome an introduced gene sequence or a removed or down-regulated nucleotide sequence, said sequence becoming up-regulated in the spinal cord of a mammal in response to first and second models of pain, particularly neuropathic or sensitisation pain, for example in response to streptozocin-induced diabetes and in response to a chronic constrictive injury to a nerve leading into the spine;

(g) an isolated polypeptide containing an amino acid sequence at least 90% identical to an amino acid sequence encoded by a nucleotide sequence according to any one of (a) to (d), or a variant thereof with sequential amino acid deletions from the C terminus and/or the N-terminus; or

(h) an isolated antibody that binds to the isolated polypeptide according to (g).

The invention further provides a compound that is useful in the treatment or diagnosis of pain and that modulates the action of an expression product of a gene sequence that becomes up-regulated in the spinal cord of a mammal in response to first and second models of pain, for example being up-regulated both in response to streptozocin induced diabetes and in response to chronic constrictive injury to a nerve leading into the spine.

The invention also relates to the use of naturally occurring compounds such as peptide ligands of the expression products of the above gene sequences and their associated signal transduction pathways for the treatment of pain.

### DESCRIPTION OF PREFERRED EMBODIMENTS

Definitions

Within the context of the present invention:

“Comprising” encompasses consisting of and including. Thus nucleic acid comprising a defined sequence includes nucleic acid that may contain a full-length gene or full-length cDNA. The gene may include any of the naturally occurring regulatory sequence(s), such as a transcription and translation start site, a promoter, a TATA box in the case of eukaryotes, and transcriptional and translational stop sites. Further, a nucleic acid sequence comprising a cDNA or gene may include any appropriate regulatory sequences for the efficient expression thereof in vitro.

“Isolated” requires that the material be removed from its original environment (e.g. the natural environment if it is naturally occurring). For example, a naturally occurring polynucleotide or a peptide present in a living animal is not isolated, but the same polynucleotide or peptide, separated from some or all of the coexisting materials in the natural system, is isolated. Such polynucleotide can be part of a vector and/or such polynucleotide or peptide can be part of a composition, and still be isolated in that the vector or composition is not a part of its natural environment.

“Mechanistically distinct” in relation to pain models implies that the pain is induced by mechanisms that differ in kind rather than being variants of a similar pain model. Thus diabetic pain and chronic constrictive pain models are mechanistically distinct whereas spinal nerve ligation models and sciatic nerve ligation models are not.

“Purified” does not require absolute purity; instead it is intended as a relative definition. Purification of starting materials or natural materials from their native environment to at least one order of magnitude, preferably two or three orders, and more preferably four or five orders of magnitude is expressly contemplated.

“Nucleic acid sequence” or “gene sequence” means a sequence of nucleotides or any variant or homologue thereof, or truncated or extended sequence thereof, and is preferably indicated by a Genebank accession number. Also within the scope of the present invention are up-regulated nucleic acid sequences which encode expression products which are components of signaling pathways. This invention also includes any variant or homologue or truncated or extended sequence of the up-regulated nucleotide sequence. Also within the scope of the present invention, the term “nucleic acid(s) product”, or “expression product” or “gene product” or a combination of these terms refers without being biased, to any, protein(s), polypeptide(s), peptide(s) or fragment(s) encoded by the up-regulated nucleotide sequence.

“Operably linked” refers to a linkage of polynucleotide elements in a functional relationship. For instance, a promoter or an enhancer is operably linked to a coding sequence if it regulates the transcription of the coding sequence. In particular, two DNA molecules (such as a polynucleotide containing a promoter region and a polynucleotide encoding a desired polypeptide) are said to be “operably linked” if the nature of the linkage between the two polynucleotides does not (1) result in the introduction of a frame-shift mutation and (2) interfere with the ability of the polynucleotide containing the promoter to direct the transcription of the coding polynucleotide.

“Gene product” refers to polypeptide—which is interchangeable with the term protein—which is encoded by a nucleotide sequence and includes single-chain polypeptide molecules as well as multiple-polypeptide complexes where individual constituent polypeptides are linked by covalent or non-covalent means.

Polypeptides of the present invention may be produced by synthetic means (e.g. as described by Geysen et al., 1996) or by recombinant means.

The terms “variant”, “homologue”, “fragment”, “analogue” or “derivative” in relation to the amino acid sequence for the polypeptide of the present invention includes any substitution of, variation of, modification of, replacement of, deletion of or addition of one (or more) amino acid from or to the sequence providing the resultant polypeptide has the native gene product activity. In particular, the term “homologue” covers homology with respect to structure and/or function. With respect to sequence homology, there is at least 90%, more preferably at least 95% homology to an amino acid sequence encoded by the relevant nucleotide sequence shown in Table 1, preferably there is at least 98% homology.

Typically, for the variant, homologue or fragment of the present invention, the types of amino acid substitutions that could be made should maintain the hydrophobicity/hydrophilicity of the amino acid sequence. Amino acid substitutions may include the use of non-naturally occurring amino acid analogues.

In addition, or in the alternative, the protein itself could be produced using chemical methods to synthesize a polypeptide, in whole or in part. For example, peptides can be synthesized by solid phase techniques, cleaved from the resin, and purified by preparative high performance liquid chromatography (e.g. Creighton (1983) Proteins Structures and Molecular Principles, W H Freeman and Co., New York, N.Y., USA). The composition of the synthetic peptides may be confirmed by amino acid analysis or sequencing (e.g. the Edman degradation procedure).

Direct peptide synthesis can be performed using various solid-phase techniques (Roberge J Y et al Science Vol 269 1995 202-204) and automated synthesis may be achieved, for example, using the ABI 431 A Peptide Synthesizer (Perkin Elmer) in accordance with the instructions provided by the manufacturer. Additionally, the amino acid sequence of a gene product, or any part thereof, may be altered during direct synthesis and/or combined using chemical methods with a sequence from other subunits, or any part thereof, to produce a variant polypeptide.

In another embodiment of the invention, a gene product natural, modified or recombinant amino acid sequence may be ligated to a heterologous sequence to encode a fusion protein. For example, for screening of libraries for compounds and peptide agonists and antagonists of gene product activity, it may be useful to encode a chimeric gene product expressing a heterologous epitope that is recognised by a commercially available antibody. A fusion protein may also be engineered to contain a cleavage site located between a gene product sequence and the heterologous protein sequence, so that the gene product may be cleaved and purified away from the heterologous moiety.

The gene product may also be expressed as a recombinant protein with one or more additional polypeptide domains added to facilitate protein purification. Such purification facilitating domains include, but are not limited to, metal chelating peptides such as histidine-tryptophan modules that allow purification on immobilised metals (Porath J, Protein Expr Purif Vol 3 1992 p 263-281), protein A domains that allow purification on immobilised immunoglobulin, and the domain utilised in the FLAGS extension/affinity purification system (Immunex Corp, Seattle, Wash., USA). The inclusion of a cleavable linker sequence such as Factor XA or enterokinase (Invitrogen, San Diego, Calif., USA) between the purification domain and the gene product is useful to facilitate purification.

“Pain” includes chronic pain and in particular diabetic pain.

“Stringent hybridization conditions” is a recognized term in the art and for a given nucleic acid sequence refers to those conditions which permit hybridization of that sequence to its complementary sequence and closely homologous nucleic acid sequences. Conditions of high stringency may be illustrated in relation to filter-bound DNA as for example 2×SCC, 65° C. (where SSC=0.15M sodium chloride, 0.015M sodium citrate, pH 7.2); or as 0.5M NaHPO₄, 7% sodium dodecyl sulfate (SDS), 1 mM EDTA, at 65° C., and washing in 0.1×SCC/0.1% SDS at 68° C. (Ausubel F. M. et al., eds, 1989, Current Protocols in Molecular Biology, Vol. I, Green Publishing Associates, Inc., and John Wiley & Sons Inc., New York, at p. 2. 10.3). Hybridization conditions can be rendered highly stringent by raising the temperature and/or by the addition of increasing amounts of formamide, to destabilize the hybrid duplex of non-homologous nucleic acid sequence relative to homologous and closely homologous nucleic acid sequences. Thus, particular hybridisation conditions can be readily manipulated, and will generally be chosen depending on the desired results.

“Variants or homologues” include (a) sequence variations of naturally existing gene(s) resulting from polymorphism(s), mutation(s), or other alteration(s) as compared to the above identified sequences, and which do not deprive the encoded protein of function (b) recombinant DNA molecules, such as cDNA molecules encoding genes indicated by the relevant Genebank accession numbers and (c) any sequence that hybridizes with the above nucleic acids under stringent conditions and encodes a functional protein or fragment thereof.

Identified Sequences

The inventors have identified nucleotide sequences that give rise to expression products listed in Table 1, that become differentially expressed in the spinal cord in response to two distinct chronic pain stimuli, for example neuropathic pain stimuli, and that are believed to be involved in the transduction of pain. In Table 1, * denotes more preferred nucleotide sequences and ** denotes most preferred nucleotide sequences. These nucleotide sequences have not previously been implicated in the transduction of pain.

The validity of the present experimental procedure was confirmed by the fact that nucleotide sequences were obtained as a result of the investigation whose function in the transduction of pain has been previously confirmed and established. These nucleic acid sequences are not part of this invention. Any of the nucleic acid sequences and expression products can be used to develop screening technologies for the identification of novel molecules for the prevention or treatment of pain. These screening technologies could also be used to ascribe new pain therapeutic indications to molecules that have not previously been identified as being useful for the prevention or treatment of pain. Furthermore, the said nucleic acid sequences can be used as diagnostic tools and for the development of diagnostic tools.

Production of Polypeptides and Nucleic Acids

Vectors

Recombinant expression vectors comprising a nucleic acid can be employed to express any of the nucleic acid sequences of the invention. The expression products derived from such vector constructs can be used to develop screening technologies for the identification of molecules that can be used to prevent or treat pain, and in the development of diagnostic tool for the identification and characterization of pain. The expression vectors may also be used for constructing transgenic non-human animals.

Gene expression requires that appropriate signals be provided in the vectors, said signals including various regulatory elements such as enhancers/promoters from viral and/or mammalian sources that drive expression of the genes or nucleotide sequences of interest in host cells. The regulatory sequences of the expression vectors used in the invention are operably linked to the nucleic acid sequence encoding the pain-associated protein of interest or a peptide fragment thereof.

Generally, recombinant expression vectors include origins of replication, selectable markers, and a promoter derived from a highly expressed gene to direct transcription of a downstream nucleotide sequence. The nucleotide sequence is assembled in an appropriate frame with the translation, initiation and termination sequences, and if applicable a leader sequence to direct the expression product to the periplasmic space, the extra-cellular medium or cell membrane.

In a specific embodiment wherein the vector is adapted for expressing desired sequences in mammalian host cells, the preferred vector will comprise an origin of replication from the desired host, a suitable promoter and an enhancer, and also any necessary ribosome binding sites, polyadenylation site, transcriptional termination sequences, and optionally 5′-flanking non-transcribed sequences. DNA sequences derived from the SV40 or CMV viral genomes, for example SV40 or CMV origin, early promoters, enhancers, and polyadenylation sites may be used to provide the required non-transcribed genetic elements.

A recombinant expression vector used in the invention advantageously also comprises an untranscribed polynucleotide region located at the 3′end of the coding sequence open reading frame (ORF), this 3′-untranslated region (UTR) polynucleotide being useful for stabilizing the corresponding mRNA or for increasing the expression rate of the vector insert if this 3′-UTR harbours regulation signal elements such as enhancer sequences.

Suitable promoter regions used in the expression vectors are chosen taking into account the host cell in which the nucleic acid sequence is to be expressed. A suitable promoter may be heterologous with respect to the nucleic acid sequence for which it controls the expression, or alternatively can be endogenous to the native polynucleotide containing the coding sequence to be expressed. Additionally, the promoter is generally heterologous with respect to the recombinant vector sequences within which the construct promoter/coding sequence has been inserted. Preferred promoters are the LacI, LacZ, T3 or T7 bacteriophage RNA polymerase promoters, the lambda P_{R}, P_{L} and Trp promoters (EP-0 036 776), the polyhedrin promoter, or the p10 protein promoter from baculovirus (kit Novagen; Smith et al., (1983); O'Reilly et al. (1992).

Preferred selectable marker genes contained in the expression recombinant vectors used in the invention for selection of transformed host cells are preferably dehydrofolate reductase or neomycin resistance for eukaryotic cell culture, TRP1 for S. cerevisiae or tetracycline, rifampicin or ampicillin resistance in E. coli, or Levamsaccharase for Mycobacteria, this latter marker being a negative selection marker.

Preferred bacterial vectors are listed hereafter as illustrative but not limitative examples: pQE70, pQE60, pQE-9 (Quiagen), pD10, phagescript, psiX174, p.Bluescript SK, pNH8A, pNH16A, pNH18A, pNH46A (Stratagene); pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIA express).

Preferred bacteriophage recombinant vectors of the invention are P1 bacteriophage vectors such as described by Sternberg N. L. (1992; 1994).

A suitable vector for the expression of any of the pain associated polypeptides used in the invention or fragments thereof, is a baculovirus vector that can be propagated in insect cells and in insect cell-lines. A specific suitable host vector system is the pVL 1392/1393 baculovirus transfer vector (Pharmingen) that is used to transfect the SF9 cell line (ATCC N°CRL 1711) that is derived from Spodoptera frugiperda.

The recombinant expression vectors of the invention may also be derived from an adenovirus. Suitable adenoviruses are described by Feldman and Steig (1996) or Ohno et al. (1994). Another preferred recombinant adenovirus is the human adenovirus type two or five (Ad 2 or Ad 5) or an adenovirus of animal origin (Patent Application WO 94/26914).

Particularly preferred retrovirus for the preparation or construction of retroviral in vitro or in vivo gene delivery vehicles include retroviruses selected from the group consisting of Mink-Cell Focus Inducing Virus, Murine Sarcoma Virus, and Ross Sarcoma Virus. Other preferred retroviral vectors are those described in Roth et al. (1996), in PCT Application WO 93/25234, in PCT Application WO 94/06920, and also in Roux et al. (1989), Julan et al. (1992) and Nada et al. (1991).

Yet, another viral vector system that is contemplated is the Adeno Associated Viruses (AAV) such as those described by Flotte et al. (1992), Samulski et al. (1989) and McLaughlin et al. (1996).

Host cells Expressing Pain Associated Polypeptides

Host cells that endogenously express pain associated polypeptides or have been transformed or transfected with one of the nucleic acid sequences described herein, or with one of the recombinant vector described above, particularly a recombinant expression vector, can be used in the present invention. Also included are host cells that are transformed (prokaryotic cells) or are transfected (eukaryotic cells) with a recombinant vector such as one of those described above.

Preferred host cells used as recipients for the expression vectors used in the invention are the following:

(a) prokaryotic host cells: Escherichia coli, strains. (i.e. DH5-α, strain) Bacillus subtilis, Salmonella typhimurium and strains from species like Pseudomonas, Streptomyces and Staphylococcus for the expression of up and down-regulated nucleic acid sequences modulated by pain, characterized by having at least 80% sequence identity with any of the nucleic acid sequences of Table 1. Plasmid propagation in these host cells can provide plasmids for transfecting other cells.

(b) eukaryotic host cells: HeLa cells (ATCC N°CCL2; N°CCL2.1; N°CCL2.2), Cv 1 cells (ATCC N°CCL70), COS cells (ATCC N°CRL 1650; N°CRL 1651), Sf-9 cells (ATCC N°CRL 1711), C127 cells (ATCC N°CRL-1804), 3T3 cells (ATCC N°CRL-6361), CHO cells (ATCC N°CCL-61), human kidney 293 cells (ATCC N° 45504; N°CRL-1573), BHK (ECACC N°84100 501; N°84111301), PC12 (ATCC N°CRL-1721), NT2, SHSY5Y (ATCC N° CRL-2266), NG108 (ECACC N°88112302) and F11, SK-N-SH (ATCC N° CRL-HTB-11), SK-N-BE (2) (ATCC N°CRL-2271), IMR-32 (ATCC N°CCL-127). A preferred system to which the nucleic acids of the invention can be expressed are neuronal cell lines such as PC 12, NT2, SHSYSY, NG108 and Fl 1, SK-N-SH, SK-N-BE (2), IMR-32 cell lines, COS cells, 3T3 cells, HeLa cells, 292 cells and CHO cells. The above cell lines could be used for the expression of any of the nucleic acid sequences of Table 1.

When a nucleic acid sequence of the Table 1 is expressed using a neuronal cell line, the sequence can be expressed under an endogenous promoter or native neuronal promoter or an exogenous promoter. Suitable exogenous promoters include SV40 and CMV and eukaryotic promoters such as the tetracycline promoter. The preferred promoter when pain associated molecules are endogenously expressed is an endogenous promoter. A preferred promoter in a recombinant cell line is the CMV promoter.

In a specific embodiment of the host cells described above, these host cells have also been transfected or transformed with a polynucleotide or a recombinant vector for the expression of a natural ligand of any of the nucleic acid sequences of Table 1 or a modulator of these expression products.

Proteins, Polypeptides and Fragments

The expression products of the nucleic acid sequences of Table I below or fragment(s) thereof can be prepared using recombinant technology, from cell lines or by chemical synthesis. Recombinant methods, chemical methods or chemical synthetic methods can be used to modify a gene in order to introduce into the gene product, or a fragment of the gene product, features such as recognition tags, cleavage sites or other modifications. For efficient polypeptide production, the endogenous expression system or recombinant expression system should allow the expression products to be expressed in a manner that will allow the production of a functional protein or fragment thereof which can be purified. Preferred cell lines are those that allow high levels of expression of polypeptide or fragments thereof. Such cell lines include cell lines which naturally express any of the nucleic acid sequences of Table 1 or common mammalian cell lines such as CHO cells or COS cells, etc, or more specific neuronal cell lines such as PC12. However, other cell types that are commonly used for recombinant protein production such as insect cells, amphibian cells such as oocytes, yeast and prokaryotic cell lines such as E. coli can also be used.

The expression products of Table 1 or fragments thereof can be utilized in screens to identify potential therapeutic ligands, either as a purified protein, as a protein chimera such as those produced by phage display, as a cell membrane (lipid or detergent) preparation, or in intact cells.

The invention also relates generally to the use of proteins, peptides and peptide fragments for the development of screening technologies for the identification of molecules for the prevention or treatment of pain, and the development of diagnostic tools for the identification and characterization of pain. These peptides include expression products of the nucleic acid sequences of Table 1 and purified or isolated polypeptides or fragments thereof having at least 90%, preferably 95%, more preferably 98% and most preferably 99% sequence identity with the any of the expression products of nucleic acid sequences of Table 1. Expressed peptides and fragments of any of these nucleic acid sequences can be used to develop screening technologies for the identification of novel molecules for the prevention or treatment of pain. These screening technologies could also be used to ascribe new pain therapeutic indications to molecules, which have not previously been ascribed for the prevention or treatment of pain. Furthermore the said expressed peptides and fragments can be used as diagnostic tools and for the development of diagnostic tools.

Screening Methods

As discussed above, we have identified nucleic acid sequences whose expression is regulated by pain, particularly chronic pain and more particularly diabetic pain. The expression products of these nucleic acids can be used for screening ligand molecules for their ability to prevent or treat pain, and particularly, but not exclusively, chronic pain. The main types of screens that can be used are described below. The test compound can be a peptide, protein or chemical entity, either alone or in combination(s), or in a mixture with any substance. The test compound may even represent a library of compounds.

The expression products of any of the nucleic acid sequences of Table 1 or fragments thereof can be utilized in a ligand binding screen format, a functional screen format or in vivo format. Examples of screening formats are provided.

A) Ligand Binding Screen

In ligand binding screening a test compound is contacted with an expression product of one of the sequences of Table 1, and the ability of said test compound to interact with said expression product is determined, e.g. the ability of the test compound(s) to bind to the expression product is determined. The expression product can be a part of an intact cell, lipidic preparation or a purified polypeptide(s), optionally attached to a support, such as beads, a column or a plate etc.

Binding of the test compound is preferably performed in the presence of a ligand to allow an assessment of the binding activity of each test compound. The ligand may be contacted with the expression product either before, simultaneously or after the test compound. The ligand should be detectable and/or quantifiable. To achieve this, the ligand can be labeled in a number of ways, for example with a chromophore, radioactive, fluorescent, phosphorescent, enzymatic or antibody label. Methods of labeling are known to those in the art. If the ligand is not directly detectable it should be amenable to detection and quantification by secondary detection, which may employ the above technologies. Alternatively the expression product or fragment thereof can be detectable or quantifiable. This can be achieved in a similar manner to that described above.

Binding of the test compound modifies the interaction of the ligand with its binding site and changes the affinity or binding of the ligand for/to its binding site. The difference between the observed amount of ligand bound relative to the theoretical maximum amount of ligand bound (or to the ligand bound in the absence of a test compound under the same conditions) is a reflection of the binding ability (and optionally the amount and/or affinity) of a test compound to bind the expression product.

Alternatively, the amount of test compound bound to the expression product can be determined by a combination of chromatography and spectroscopy. This can also be achieved with technologies such as Biacore (Amersham Pharmacia). The amount of test compound bound to the expression product can also be determined by direct measurement of the change in mass upon compound or ligand binding to the expression product. Alternatively, the expression product, compound or ligand can be fluorescently labelled and the association of expression product with the test compound can be followed by changes in Fluorescence Energy Transfer (FRET).

The invention therefore includes a method of screening for pain alleviating compounds, comprising:

a) contacting a test compound or test compounds in the presence of a ligand with an expression product of any of the nucleic acid sequences of Table 1 or with cell expressing at least one copy of the expression product or with a lipidic matrix containing at least one copy of the expression product; determining the binding of the test compound to the expression product, and

b) selecting test compounds on the basis of their binding abilities.

In the above method, the ligand may be added prior to, simultaneously with or after contact of the test compound with the expression product. Non limiting examples and methodology can be gained from the teachings of the Molecular Probes handbook and references therein (Molecular Probes, Inc., 4849 Pitchford Ave, Eugene, USA), Methods in neurotransmitter receptor analysis (Yamamura H I., Enna, S J., and Kuhar, M J., Raven Press New York, the Glaxo Pocket Guide to Pharmacology, Dr. Michael Sheehan, Glaxo Group Research Ltd, Ware, Herts SG12 ODP), Bylund D B and Murrin L C (2000, Life Sciences, 67 (24) 2897-911), Owicki J C (2000, J. biomol Screen (5) 297-306), Alberts et al (1994, Molecular Biology of the Cell, 3^{rd} Edn, Garland Publication Inc), Butler J E, (2000 Methods 22(1):4-23, Sanberg S A (2000, Curr Opin Biotechnol 11(1) 47-53), and Christopoulos A (1999, biochem Pharmacol 58(5) 735-48).

Functional Screening

(a) Kinase Assays

The expression products of any of the nucleic acid sequences of Table 1 which encode a kinase are amenable to screening using kinase assay technology.

Kinases have the ability to add phosphate molecules to specific residues in ligands such as binding peptides in the presence of a substrate such as adenosine triphosphate (ATP). Formation of a complex between the kinase, the ligand and substrate results in the transfer of a phosphate group from the substrate to the ligand. Compounds that modulate the activity of the kinase can be determined with a kinase functional screen. Functional screening for modulators of kinase activity therefore involves contacting one or more test compounds with an expression product of one of the nucleic acid sequences of Table 1 which encodes a kinase, and determining the ability of said test compound to modulate the transfer of a phosphate group from the substrate to the ligand.

The expression product can be part of an intact cell or of a lipidic preparation or it can be a purified polypeptide(s), optionally attached to a support, for example beads, a column, or a plate. Binding is preferably performed in the presence of ligand and substrate to allow an assessment of the binding activity of each test compound.

The ligand should contain a specific kinase recognition sequence and it should not be phosphorylated at its phosphoryation site. The ligand and/or substrate may be contacted with the kinase either before, simultaneously or after the test compound. Optionally the substrate may be labelled with a kinase transferable labelled phosphate. The assay is monitored by the phosphorylation state of the substrate and/or the ligand. The ligand should be such that its phosphorylation state can be determined. An alternative method to do this is to label the ligand with a phosphorylation-state-sensitive molecule. To achieve this, the ligand can be labelled in a number of ways, for example with a chromophore, radioactive, fluorescent, phosphorescent, enzymatic or antibody label. If the ligand is not directly detectable it should be amenable to detection and quantification by secondary detection, which may employ the above technologies. Such technologies are known to those in the art.

Binding of the test compound to the kinase modifies its ability to transfer a phosphate group from the substrate to the ligand. The difference between the observed amount of phosphate transfer relative to the theoretical maximum amount of phosphate transfer is a reflection of the modulatory effect of the test compound. Alternatively, the degree of phosphate transfer can be determined by a combination of chromatography and spectroscopy. The extent of phosphorylation of the ligand peptide or dephosphorylation of the substrate can also be determined by direct measurement. This can be achieved with technologies such as Biacore (Amersham Pharmacia).

The invention also provides a method for screening compounds for the ability to relieve pain, which comprises:

(a) contacting one or more test compounds in the presence of ligand and substrate with an expression product of any of the nucleotide sequences of Table I which is a kinase or with a cell containing at least 1 copy of the expression product or with a lipidic matrix containing at least 1 copy of an expression product;

(b) determining the amount of phosphate transfer from the substrate to the ligand; and

(c) selecting test compounds on the basis of their capacity to modulate phosphate transfer.

Optionally ligand, substrate and/or other essential molecules may be added prior to contacting the test compound with expression product of step (a) or after step (a). Non limiting examples and methodology can be gained from the teachings of the Molecular Probes handbook and references therein (Molecular Probes, Inc., 4849 Pitchford Ave, Eugene, USA), Methods in Molecular Biology 2000; 99: 191-201, Oncogene 2000 20; 19(49): 5690-701, and FASAB Journal, (10, 6, P55, P1458, 1996, Pocius D Amrein K et al).

b) Phosphatase Assays

The expression products of any of the nucleic acid sequences of Table 1 which encode a phosphatase are amenable to screening using phosphatase assay technology.

Phosphatase enzymes have the ability to remove phosphate molecules from specific residues in ligands such as peptides. This reaction takes place in the presence of a substrate such as Adenosine Diphosphate (ADP). The complexing of the phosphatase polypeptide, ligand and substrate results in the transfer of a phosphate group from the ligand to substrate. Compounds that modulate the activity of the Phosphatase can be determined with a Phosphatase functional screen. This screen detects the reverse of the Kinase functional screen outlined above.

The invention also provides a method for screening compounds for the ability to relieve pain, which comprises:

(a) contacting at one or more test compounds in the presence of ligand and substrate with an expression product of any of the nucleotide sequences of Table 1 which is a phosphatase or with a cell containing at least 1 copy of the expression product or with a lipidic matrix containing at least 1 copy of an expression product;

(b) determining the amount of phosphate transfer from the ligand to the substrate; and

(c) selecting test compounds on the basis of their capacity to modulate phosphate transfer.

Optionally ligand, substrate and/or other essential molecules may be added prior to contacting the test compound with expression product of step (a) or after step (a). Non-limiting examples and methodology can be gained from the teachings of the Molecular Probes handbook and references therein (Molecular Probes, Inc., 4849 Pitchford Ave, Eugene, USA), and FASAB Journal, (10, 6, P55, P1458, 1996, Pocius D Amrein K et al).

c) Phosphodiesterase Assays

An expression product of any of the nucleic acid sequences of Table I which encodes a phosphodiesterase is amenable to screening using phosphodiesterase assay technology.

Phosphodiesterases have the ability to cleave cyclic nucleotides cAMP (cyclic adenosine monophosphate) and/or cGMP (cyclic guanosine monophosphate) (substrate) at their 3′phosphatase bond to form 5′AMP and 5′GMP. Functional screening for modulators of phosphodiesterase polypeptide comprises contacting one or more test compounds with an expression product as set out above which is a phosphodiesterase and determining the ability of said test compound(s) to modulate the cleavage of cyclic nucleotides cAMP and/or cGMP at their 3′phosphatase bond to form 5′AMP and 5′GMP. The expression product can be part of an intact cell or lipidic preparation or a purified polypeptide(s), optionally attached to a support, for example beads, a column, or a plate. Binding is preferably performed in the presence of cAMP or cGMP to allow an assessment of the binding activity of each test compound. The cAMP or cGMP and other essential molecules may be contacted with the phosphodiesterase peptide either before, simultaneously or after the test compound(s).

A characteristic of the cAMP or cGMP is that it can readily be radio labeled (Thompson et al, Advances in cyclic nucleotide research, 10, 69-92 (1974)). The conversion of cAMP or cGMP to 5′AMP or 5′GMP can be detected with the use of chromatography and separation technologies. Such technology is known to those in the art. Binding of the test compound to the phosphodiesterase polypeptide modifies its ability to convert cAMP or cGMP to 5′AMP or 5′GMP. The difference between the observed amount of conversion relative to the theoretical maximum amount of conversion is a reflection of the modulatory effect of the test compound(s).

A non-limiting general understanding of how to assay for the activity of Phosphodiesterases can be gained from Horton, J K. And Baxendale, P M (Methods in molecular Biology, 1995, 41, p 91-105, Eds. Kendall, D A. and Hill, S J, Humana Press, Towota, N.J.) and Molecular Probes handbook and references therein (Molecular Probes, Inc., 4849 Pitchford Ave, Eugene, USA).

The invention also provides methods of screening for pain alleviating compounds, comprising;

a) contacting one or more test compounds in the presence of cAMP and/or cGMP with an expression product of any of the nucleotide sequences of Table 1 which is a phosphodiesterase or with a cell expressing at least 1 copy of an expression product or with a lipidic matrix containing at least 1 copy of an expression product;

b) determining the amount of cAMP and/or cGMP converted to 5′AMP and/or 5′GMP, and

c) selecting test compounds on the basis of their ability to modulate said conversion.

d) Ion Channel Protein Assays

An expression product of any of the nucleic acid sequences of Table 1 which encodes an ion channel protein, and in particular any of the nucleic acid sequences listed in Table 1, is amenable to screening using ion channel protein assay technology.

Ion channels are membrane associated proteins. They are divided into three main groups:

(1) ligand gated ion channels;

(2) voltage gated ion channels; and

(3) mechano gated ion channels.

Ion channels allow the passage of ions through cellular membranes upon stimulation by ligand, change in membrane potential or physical changes in environment such as temperature and pH. Compounds that modulate the activity of ion channels can be determined with an ion channel functional screen.

Functional screening for modulators of ion channels involves contacting a test compound with an expression product as aforesaid which is an ion channel protein or a fragment thereof and determining the ability of said test compound to modulate the activity of said expression product or fragment thereof. The expression product can be a part of an intact cell, membrane preparation or lipidic preparation, optionally attached to a support, for example beads, a column, or a plate. The ligand may be contacted with the ion channel peptide before, simultaneously with or after the test compound. Optionally other molecules essential for the function of the ion channel may be present. Ion channel opening is detectable with the addition of Ion channel sensitive dye, such dyes are known to those in the art. Examples are provided in Molecular Probes handbook and references therein (Molecular Probes, Inc., 4849 Pitchford Ave, Eugene, USA) and Glaxo Pocket Guide to Pharmacology (Dr Michael Sheenal Pharmacology Division Staff, Glaxo Group Research Ltd., Ware, Herts SG12 0DP). Binding of the test compound to the ion channel protein modifies its ability to allow ion molecules across a membrane. The difference between the observed amount of movement of ions across a membrane relative to the theoretical maximum amount of ions that can move across the membrane is a reflection of the modulatory effect of the test compound.

The invention therefore also relates to a method of screening compounds for their ability to alleviate pain, which method comprises:

(a) contacting at least one test compound in the presence of voltage potential sensing dye with a cell containing at least 1 copy of an expression product of any of the nucleotide sequences of Table 1 which is an ion channel protein or with a lipidic matrix containing at least one copy of the expression product;

(a) applying a ligand or stimulus;

(b) determining the opening of the ion channel, and

(c) selecting a test compound on the basis of its ability to modulate movement of ions across the membrane.

e) Receptor Assays

An expression product of any of the nucleic acid sequences of Table 1 which encodes a receptor is amenable to screening using receptor assay technology.

Receptors are membrane associated proteins that initiate intracellular signalling upon ligand binding. Therefore, the identification of molecules for the prevention and treatment of pain can be achieved with the use of a ligand-binding assay, as outlined above. Such an assay would utilize an endogenous or non-endogenous ligand as a component of the ligand-binding assay. The binding of this ligand to the receptor in the presence of one or more test compounds would be measured as described above. Such is the nature of receptors that the assay is usually, but not exclusively performed with a receptor as an intact cell or membranous preparation.

The invention therefore includes a method of screening for pain alleviating compounds, comprising:

(a) contacting a test compound or test compounds in the presence of a ligand with cells expressing at least one copy of the expression product of any of the nucleotide sequences of Table 1 which is a receptor or with a lipidic matrix containing at least one copy of the expression product;

(b) determining the binding of the test compound to the expression product, and

(c) selecting test compounds on the basis of their binding abilities.

Transporter Protein Assays

An expression product of any of the nucleic acid sequences of Table 1 which encodes a transporter protein which is amenable to screening using transporter protein assay technology. Non limiting examples of technologies and methodologies are given by Carroll F I, et al (1995, Medical Research Review, Sep. 15 (5) p 419-444), Veldhuis J D and Johnson Ml (1994, Neurosci. Biobehav Rep., winter 18(4) 605-12), Hediger M A and Nussberger S (1995, Expt Nephrol, July-August 3(4) p 211-218, Endou H and Kanai Y, (1999, Nippon Yakurigaku Zasshi, October 114 Suppl 1:1p-16p), Olivier B et al (2000, Prog. Drug Res., 54, 59-119), Braun A et al (2000, Eur J Pharm Sci, October 11, Supply 2 S51-60) and Molecular Probes handbook and references therein (Molecular Probes, Inc., 4849 Pitchford Ave, Eugene, USA).

The main function of transporter proteins is to facilitate the movement of molecules across a cellular membrane. Compounds that modulate the activity of transporter proteins can be determined with a transporter protein functional screen. Functional screening for modulators of transporter proteins comprises contacting at least one test compound with an expression product as aforesaid which is a transporter protein and determining the ability of said test compound to modulate the activity of said transporter protein. The expression product can be part of an intact cell, or lipidic preparation, optionally attached to a support, for example beads, a column or a plate. Binding is preferably performed in the presence of the molecule to be transported, which should only able to pass through a cell membrane or lipidic matrix with the aid of the transporter protein. The molecule to be transported should be able to be followed when it moves into a cell or through a lipidic matrix. Preferably the molecule to be transported is labelled to aid in characterization, e.g. with a chromophore, radioactive, fluorescent, phosphorescent, enzymatic or antibody label. If the molecule to be transported is not directly detectable it should be amenable to detection and quantification by secondary detection, which may employ the above technologies. The molecule to be transported may be contacted with the transporter protein before, simultaneously with or after the test compound. If the binding of the test compound to the transporter protein modifies its ability to transport molecules through a membranous or lipidic matrix, then the difference between the observed amount of transported molecule in a cell/or through a lipidic matrix relative to the theoretical maximum amount is a reflection of the modulatory effect of the test compound.

The invention further provides a method for screening compounds for their ability to relieve pain, comprising

a) contacting at least one test compound in the presence of transporter molecules with a cell containing at least one copy of an expression product of any of the nucleotide sequences of Table 1 which is a transporter protein or with a lipidic matrix containing at least one copy of the expression product;

c) measuring the movement of transported molecules into or from the cell, or across the lipidic matrix; and

d) selecting test compounds on the basis of their ability to modulate the movement of transported molecules.

DNA-Binding Protein Assays

An expression product of any of the nucleic acid sequences of Table 1 that encodes a DNA-binding protein is amenable to screening using DNA-binding protein assay technology.

DNA binding proteins are proteins that are able to complex with DNA. The complexing of the DNA binding protein with the DNA in some instances requires a specific nucleic acid sequence. Screens can be developed in a similar manner to ligand binding screens as previously indicated and will utilise DNA as the ligand. DNA-binding protein assays can be carried using similar principles described in ligand binding assays as described above. Non limiting examples of methodology and technology can be found in the teachings of Haukanes B I and Kvam C (Biotechnology, Jan 11, 1993 60-63), Alberts B et al (Molecular Biology of the Cell, 1994, 3^{rd} Edn., Garland Publications Inc, Kirigiti P and Machida C A (2000 Methods Mol Biol, 126, 431-51) and Molecular Probes handbook and references therein (Molecular Probes, Inc., 4849 Pitchford Ave., Eugene, USA).

The invention therefore includes a method of screening for pain alleviating compounds, comprising:

(a) contacting a test compound or test compounds in the presence of a plurality of nucleic acid sequences with an expression product of any of the nucleic acid sequences of Table 1 which is a DNA binding protein or with cell expressing at least one copy of the expression product or with a lipidic matrix containing at least one copy of the expression product;

(b) determining the binding of the test compound to the expression product, and

(c) selecting test compounds on the basis of their binding abilities.

In the above method, the plurality of nucleic acid sequence may be added prior to, simultaneously with or after contact of the test compound with the expression product.

Oxidoreductases

Oxidoreductases are enzymes that catalyse the transfer of hydrogen or oxygen atoms or electrons. These enzymes can by sub-grouped into twenty categories according to their specific mode of action. These groups are oxidoreductases acting on the CH—OH group of donors (E.C. No 1.1), oxidoreductases acting on the aldehyde or oxo group of donors (E.C. No 1.2), oxidoreductases acting on the CH—CH group of the donor (E.C. No 1.3), oxidoreductases acting on the CH-NH2 group of donors (E.C. 1.4), oxidoreductases acting on the CH—NH group of donor (E.C. 1.5), oxidoreductases acting on NADH or NADPH (E.C. No 1.6), oxidoreductases acting on other nitrogen compounds as donors (E.C. No 1.7), oxidoreductases acting on a sulphur group of donors (E.C. No 1.8), oxidoreductases acting on a haem group of donors (E.C. No 1.9), oxidoreductases acting on diphenols and related substances as donors (E.C. 1.10), oxidoreductases acting on hydrogen peroxide as acceptor (E.C. No 1.11), oxidoreductases acting on hydrogen as donor (E.C. No 1.12), oxidoreductases acting on single donors with incorporation of molecular oxygen (E.C. No 1.13), oxidoreductases acting on paired donors with incorporation of molecular oxygen (E.C. No 1.14), oxidoreductases acting on superoxide radicals as acceptors (E.C. 1.15), oxidoreductases oxidizing metal ions (E.C. No 1.16), oxidoreductases acting on -CH2 groups (E.C. No 1.17), oxidoreductases acting on reduced ferredoxin as donor (E.C. No 1.18), oxidoreductases acting on reduced flavodoxin as donor (E.C. No 1.19) and other oxidoreductases (E.C. No 1.97) (Analytical Biochemistry 3^{rd} Edn, David J. Holme and Hazel Peck, Longman Press). The enzyme commission number (E.C.) of the International Union of Biochemistry relates to the type of reaction catalysed by the enzyme. Further teachings on how to develop assays and screens for oxidoreductases can be obtained from Methods in Enzymology (Academic Press) with special reference to volume 249.

Hydrolases

Hydrolases are enzymes that catalyse hydrolytic reactions and are sub-grouped into eleven classes according to the type of reaction they carry out. Hydrolases acting on ester bonds (E.C. No 3.1), hydrolases acting on glycosyl compounds (E.C. No 3.2), hydrolases acting on ether bonds (E.C. No 3.3), hydrolases acting on peptide bonds (E.C. No 3.4), hydrolases acting on carbon-nitrogen bonds, other than peptide bonds (E.C. No 3.5), hydrolases acting on acid anhydrides (E.C. No 3.6), hydrolases acting on acid anhydrides (E.C. No 3.6), hydrolases acting on carbon-carbon bonds (E.C. No 3.7), hydrolases acting on halide bonds (E.C. No 3.8), hydrolases acting on phosphorous-nitrogen bonds (E.C. No 3.9), hydrolases acting on sulphur-nitrogen bonds (E.C. No 3.10) and hydrolases acting on carbon-phosphorous bonds (Analytical Biochemistry 3^{rd} Edn, David J. Holme and Hazel Peck, Longman Press). The enzyme commission number (E.C.) of the International Union of Biochemistry relates to the type of reaction catalysed by the enzyme. Further teachings on how to develop assays and screens for hydrolases can be obtained from Methods in Enzymology (Academic Press) with special reference to volume 249.

C) In vivo Functional Screen

Any of the nucleotide sequences of Table 1 or homologues thereof may be inserted by means of an appropriate vector into the genome of a lower vertebrate or of an invertebrate animal or may be inactivated or down regulated in the genome of said animal. The resulting genetically modified animal may be used for screening compounds for effectiveness in the regulation of pain. The invertebrate may, for example, be a nematode e.g. Caenorhabditis elegans, which is a favourable organism for the study of response to noxious stimuli. Its genome sequence has been determined, see Science, 282, 2012 (1998), it can be bred and handled with the speed of a micro-organism (it is a self-fertilizing hermaphrodite) and can therefore be used in a high throughput screening format (WO 00/63424, WO 00/63425, WO 00/63426 and WO 00/63427), and it offers a full set of organ systems, including a simple nervous system and contains many similarly functioning genes and signaling pathways to mammals. A thermal avoidance model based on a reflexive withdrawal reaction to an acute heat stimulus has been described by Wittenburg et al, Proc. Natl. Acad. Sci. USA, 96, 10477-10482 (1999), and allows the screening of compounds for the treatment of pain with the modulation of pain sensation as an endpoint.

The genome of C. elegans can be manipulated using homologous recombination technology which allows direct replacement of nucleic acids encoding C. elegans with their identified mammalian counterpart. Replacement of these nucleic acids with those nucleic acids outlined above would allow for the direct screening of test compound(s) with their expression products. Any of the pain-related genes described above may be ligated into a plasmid and introduced into oocytes of the worm by microinjection to produce germline transformants. Successful plasmid injection into C. elegans and expression of inserted sequences has been reported by Devgen B. V., Ghent, Belgium. It is also possible to produce by routine methods worms in which the target sequences are down-regulated or not expressed (knock-out worms). Further non limiting examples of methodology and technology can be found in the teachings of Hazendonk et al (1997, Nat genet. 17(1) 119-21), Alberts et al, (1994, Molecular Biology of the Cell 3^{rd} Ed. Garland Publishing Inc, Caenorhabditis elegans is anatomically and genetically simple), Broverman Set al, (1993, PNAS USA 15; 90(10) 4359-63) and Mello et al (1991, 10(12) 3959-70).

A further method for screening compounds for ability to modify response to pain, e.g. relieve pain, comprises:

(a) contacting one or more test compounds with at least one C elegans containing at least one copy of a sequence as set out above;

(b) subjecting the C. elegans to a nociceptive stimulus;

(c) observing the response of the C. elegans to said stimulus; and

(d) selecting test compounds on the basis of their ability to modify the response of C. elegans to said stimulus.

Diagnostic Tools and Kits

Affinity Peptides, Ligands and Substrates

Pain associated polypeptides and fragments thereof can be detected at the tissue and cellular levels with the use of affinity peptides, ligands and substrates, which will enable a skilled person to define more precisely a patient's ailment and help in the prescription of a medicament. Such affinity peptides are characterized in that firstly they are able to bind specifically to a pain associated polypeptide, and secondly that they are capable of being detected. Such peptides can take the form of a peptide or polypeptide for example an antibody domain or fragment, or a peptide/polypeptide ligand or substrate, or a polypeptide complex such as an antibody.

The preparation of such peptides and polypeptides are known to those in the art. Antibodies, these may be polyclonal or monoclonal, and include antibodies derived from immunized animals or from hybridomas, or derivatives thereof such as humanized antibodies, Fab or F(ab′)2 antibody fragments or any other antibody fragment retaining the antigen binding specificity.

Antibodies directed against pain-associated gene product molecules may be produced according to conventional techniques, including the immunization of a suitable mammal with the peptides or polypeptides or fragment thereof. Polyclonal antibodies can be obtained directly from the serum of immunized animals. Monoclonal antibodies are usually produced from hybridomas, resulting from a fusion between splenocytes of immunized animals and an immortalized cell line (such as a myeloma). Fragments of said antibodies can be produced by protease cleavage, according to known techniques. Single chain antibodies can be prepared according to the techniques described in U.S. Pat. No. 4,946,778. Detection of these affinity peptides could be achieved by labeling. Technologies, which allow detection of peptides, such as enzymatic labeling, fluorescence labeling or radiolabeling are well known to those in the art. Optionally these affinity peptides, ligands and substrates could themselves be detected with the use of a molecule that has specific affinity to the peptides, ligands and substrates and is itself labeled.

The invention further provides a kit comprising;

(a) affinity peptide and/or ligand and/or substrate for an expression product of a gene sequence that is up-regulated in the spinal cord of a mammal in response to first and second models of neuropathic or central sensitization pain; and

(b) a defined quantity of an expression product of a gene sequence that is up-regulated in the spinal cord of a mammal both in response to first and second models of neuropathic or central sensitization pain,

for simultaneous, separate or sequential use in detecting and/or quantifying an expression product of a gene sequence that is up-regulated in the spinal cord of a mammal in response to first and second models of neuropathic or central sensitization pain.

Complimentary nucleic acids

Pain associated nucleic acid sequences can be characterized at the tissue and cellular levels with the use of complimentary nucleic acid sequences. Detection of the level of expression of pain associated nucleic acid sequences can help in the prognosis of a pain condition and the prescription of a medicament. These complimentary nucleic acids are characterized in that they can hybridize to a pain associated nucleic acid sequence and their presence can be detected through various techniques. Such techniques are known to those in the art and may include detection by polymerase chain reaction or detection by labeling of complimentary nucleic acid sequences by enzymatic labeling, affinity labeling fluorescent labeling or radiolabeling. Complimentary strand nucleic acid sequences of this invention are 10 to 50 bases long, more preferably 15 to 50 bases long and most preferably 15 to 30 bases long, and hybridize to the coding sequence of the nucleic acid sequences.

A further aspect of this invention is a kit that comprises:

(a) nucleic acid sequences capable of hybridization to a nucleic acid sequence that is up-regulated in the spinal cord of a mammal in response to first and second models of neuropathic or central sensitization pain; and

(b) a defined quantity of one or more nucleic acid sequences capable of hybridization to a nucleic acid sequence that is up-regulated in the spinal cord of a mammal in response to first and second models of neuropathic or central sensitization pain, for simultaneous, separate or sequential use in detecting and/or quantifying a gene sequence that is up-regulated in the spinal cord of a mammal in response to first and second models of neuropathic or central sensitization pain.

Identification and Validation

Subtractive hybridization enables the identification of nucleic acid sequences whose expression profiles are modified by a stimulus. Upon stimulation of a system (in the case of this invention a nociceptive stimulus on an animal model) all observed changes in the level of nucleic acid sequence expression are due to the reaction of the system to the stimulus. Characterization of these changes in expression by way of identification of nucleic acid sequence and level of expression is both identification and validation.

The inventors have developed a four step process which allows for the simultaneous identification and validation of nucleic acid sequences whose expression are regulated by a pain stimulus, preferably a chronic pain stimulus, and more preferably a diabetic pain stimulus. This process may comprise the following steps:

(a) induction of a nociceptive stimulus in test animals;

(b) extraction of nucleic acids from specific neuronal tissue of test and control animals;

(c) selective amplification of differentially expressed nucleic acid sequence; and

(d) identification and characterization of differentially expressed gene products that are modulated by a nociceptive stimulus.

The above process is described in more detail below.

(a) Induction of Nociceptive Stimulus

The effect of the selected nociceptive stimulus on the test animal needs to be confirmable. The test subjects are therefore a species that has a “developed” nervous system, preferably similar to that of humans, most preferably rats or mice. Advantageously, the nociceptive stimulus is analogous to known pain paradigms in humans. One such paradigm of pain is the pain associated with diabetes, which can be induced in rodents with the use of streptozotocin (STZ). The present application requires the sequences to be up-regulated in two pain models, and mechanical damage can provide an appropriate second model.

Streptozotocin (STZ) induces hyperglycemia and Type 1 diabetes mellitus in rats. In particular, STZ contains a glucose analogue that allows it to be taken up by the glucose transporter 2 present on the surface of pancreatic β cells, the site of insulin synthesis. Once inside the cell, STZ causes a reduction in the level of nicotinamide adenine dinucleotide (NAD⁺). The decrease in NAD⁺ levels eventually leads to necrosis of the pancreatic β cell, causing a reduction in insulin levels and then diabetes, leading to neuropathy (diabetic) and neuropathic pain (R. B. Weiss, Cancer Treat. Rep., 66, 427-438 1982, Guy et al, Diabetologica, 28, 131-137 1985; Ziegler et al, Pain, 34, 1-10 1988; Archer et al, J. Neural. Neurosurgeon. Psychiatry, 46, 491-499 1983). The diabetic rat model has been shown to be a reliable model of hyperalgesia. We have used an STZ-induced diabetic rat model to create a state of hyperlagesia that can be compared with control animals (Courteix et al, Pain, 53, 81-88 1993).

Three models of neuropathic pain in rats, which involve nerve injury, may be used, see Ralston, D D (1998) Present models of neuropathic pain. Pain Rev. 5: 83-100. The injuries are caused by (1) loosely tying four chromic gut sutures around the sciatic nerve (CCI model developed by Bennett, G J and Y-K Xie, A peripheral mononeuropathy in rat that produces disorders of pain sensation like those seen in man, Pain 33: 87-107, 1988), (2) tightly ligating one third to one half of the fibers in the sciatic nerve (model developed by Seltzer, Z, R Dubner, Y Shire, A novel behavioral model of neuropathic pain disorders produced in rats by partial sciatic nerve injury, Pain 43: 205-218, 1990), and (3) tightly ligating the dorsal spinal nerve of a rat at the L5 or L5 and L6 levels (L5 model developed by Kim, S H and J M Chung, An experimental model for peripheral neuropathy produced by segmental spinal nerve ligation in the rat, Pain 50:355-363, 1992).

(b) Extraction of Nucleic Acids from Neuronal Tissue of Test and Control Animals

The inventors have determined that RNA extraction of whole spinal cord nervous tissue would provide a way of identifying nucleic acid sequences whose expression in spinal tissue is modulated by streptozotocin induced diabetes or by a mechanical nerve damage model for neuropathic pain e.g. CCI. Test (subjected to the nociceptive stimulus) and control animals were sacrificed, and the tissue to be studied e.g. neural tissue separated. Techniques for so doing vary widely from animal to animal and will be familiar to skilled persons.

A cDNA library can be prepared from total RNA extracted from neural tissue of the test and control animals. Where possible, however, it is preferred to isolate the mRNA from the total RNA of the test and control animals, by affinity chromatography on oligo (dT)-cellulose, and then reverse transcribe the mRNA from the test and control animals to give test and control cDNA. Converting mRNA from the test and control animals to corresponding cDNA may be carried out by any suitable reverse transcription method, e.g. a method as described by Gubler & Hoffman, Gene, 25, 263-269 (1983). If desired a proprietary kit may be used e.g. the CapFinder PCR cDNA Library Construction Kit (Life Technologies) which is based on long-distance PCR and permits the construction of cDNA libraries from nanograms of total RNA.

(c) Selective Amplification of Differentially Expressed Nucleic Acids

The reverse transcribed cDNA of the test and control animals is subjected to subtractive hybridisation and amplification so that differentially expressed sequences become selectively amplified and commonly expressed sequences become suppressed, so as to over-produce DNA associated with said nociceptic stimulus. A wide range of subtractive hybridisation methods can be used, but the preferred method is so-called suppression subtractive hybridisation, see U.S. Pat. No. 5,565,340 and Diatchenko et al, Proc. Nat. Acad. Sci. USA, 93, 6025-6030 (1996), the disclosures of which are herein incorporated by reference. Kits for carrying out this method are available from CLONTECH Laboratories, Inc.

(d) Cloning and Sequencing the Differentially Expressed cDNA

The differentially expressed cDNA is ligated into a cloning vector, after which cells of E. coli are transformed with the vector and cultured. Positive clones are selected and lysed to release plasmids containing the cDNA insert. The plasmids are primed using forward and reverse primers to either side of the cloning site and the cDNA insert is sequenced. Vector and adaptor sequences are then removed from the output data from the sequencer, leaving only the nucleotide sequence of the differentially expressed gene. The sequence is then checked against data held in a database for homology to known nucleotide sequences including expressed sequence tags (ESTs) and coding sequences for proteins.

(e) Validation of the Above Method

The importance of the sequences that we have identified in pain is confirmed by the fact that genes that represent nucleic acid sequences which have previously been implicated in pain, including Calmodulin (pRCM1, Genebank X13933), Enkephalin (Genebank Y07503) and Neurotensin receptor type 2 (Genebank X97121) have also been identified using this method.

The inventors have identified nucleic acid sequences of the MAP kinase pathway, a previously non pain-associated biological pathway. The inventors have subsequently shown that intra-spinal injection of a MEK inhibitor (MEK is part of the MAP kinase pathway) produces a powerful inhibition of pain (Patent application No U.S. 60/144,292). Subsequently, it has been shown that the MAP kinase is also implicated in acute inflammatory pain (Woolf et al, Nature Neuroscience 1999).

The invention will now be further described in the following Example.

### EXAMPLE

Induction of Diabetes

Diabetes was induced in adult (150-200 g) male Sprague-Dawley rats (n=6) as described by Courteix et al (supra). Animals were injected intraperitoneally with streptozotocin (STZ)(50 mg/kg) dissolved in distilled water. Control or sham animals (age-matched animals, n=6) were injected with distilled water only.

Chronic Constrictive Injury (CCI)

Rats were anaesthetized with i.p. sodium phenobarbital, after which the common left sciatic nerve was exposed at the level of the middle of the thigh by blunt dissection through the biceps femoris and proximal to the sciatic trifurcation. Four ligatures (4.0 braided silk) were tied loosely around it with about 1 mm spacing. The muscle was closed in layers and two wound clips were applied to close the skin incision. The wound was then covered with topical antibiotics.

Nociceptive Testing

Static allodynia (a form of hyperlagesia) was measured using a method described by Chaplan let al, “Quantitative assessment of tactile allodinya in the rat paw”, J. Neurosci. Methods, 53, 55-63 (1994). A series of von Frey filaments of different buckling weight (i.e. the load required for the filament to bend) were applied to the plantar surface of the right hand paw. The starting filament had a buckling weight of 20 g. Lifting of the paw was taken to be a positive result, in which case a filament with the next lowest buckling weight was used for the next measurement. The test was continued until a filament was found for which there was an absence of response for longer than 5 seconds whereas a re-test with the next heaviest filament gave a positive response. Animals were considered hyperalgesic if their thresholds were found to be <4 g of those of comparable untreated rats, see Calcutt & Chaplan, “Spinal pharmacology of tactile allodynia in diabetic rats”, British J. Pharmacol, 122, 1478-1482 (1997).

Tissue Extraction

STZ-treated, CCI-treated and control animals were anaesthetized with 4% halothane and perfused with ice-cold 0.9% saline containing 1% citric acid (pH adjusted to 7.4 with NaOH). The animals were decapitated and the lumbar spinal cord exposed. A 2-centimetre length of spinal cord ending at L6 (lumbar-6 forward) was removed from the spinal column. Attached dorsal root ganglia and contaminating spinal connective tissues were removed. The spinal cord tissue was snap frozen in dry ice and isopentane. In the experiments that follow, procedures on the streptozocin-treated and control groups of animals are disclosed. It will be understood that for identification of CCI-treated animals the same experiments are performed, but using tissues from the CCI-treated animals and from control animals.

Total RNA Extraction

Total RNA was extracted from the pooled male rat tissues of the streptozocin-treated and control groups using the TRIZOL Reagent Kit (Life Technologies). Briefly, tissue samples were homogenised fully using a Polytron homogenizer in 1 ml of TRIZOL reagent per 50-100 mg of tissue. Homogenized samples were then incubated at room temperature for 5 minutes and phase separated using 0.2 ml chloroform per 1 ml TRIZOL reagent followed by centrifugation at 3,000 g. The aqueous phase was transferred to a fresh tube and the RNA precipitated with an equal volume of isopropyl alcohol and followed by centrifugation at 10,000 g. The RNA pellet was washed in 75% ethanol and re-centrifuged. The pellet was then air dried and re-suspended in water.

mRNA Extraction

In contrast to ribosomal RNA and transfer RNA, the vast majority of mRNAs of mammalian cells carry tracts of poly(A+) at their 3′ termini. mRNAs can therefore be separated from the bulk of cellular RNA by affinity chromatography on oligo (dT)-cellulose. mRNA was extracted from Total RNA using the MESSAGEMAKER Kit (Life Technologies) in which mRNA (previously heated to 65° C. in order to disrupt secondary structures and so expose the poly (A⁺) tails) was bound to oligo (dT) cellulose under high salt concentrations (0.5M NaCl) in a filter syringe. Unbound RNA was then washed away and the poly(A⁺) mRNA eluted in distilled water. A tenth of the volume of 7.5 M Ammonium Acetate, 50 μg of glycogen/ml mRNA sample and 2 volumes of absolute alcohol were then added to the samples which are then placed at −20° C. overnight. Following precipitation, the mRNA was spun down at 12,000 g for 30 minutes at 4° C. RNAase free water was used to re-suspend the pellets, which were then and stored at −80° C.

PCR Select

The technique used was based on that of the CLONTECH PCR Select Subtraction Kit. The following protocol was performed using STZ-treated lumbar spinal cord Poly A⁺ RNA as the ‘Tester’ and Sham lumbar spinal cord poly A⁺ RNA as the ‘Driver’ (Forward Subtraction). A second subtraction experiment using the Sham lumbar spinal cord mRNA as the ‘Tester’ and STZ treated lumbar spinal cord mRNA as the ‘Driver’ (Reverse Subtraction) was performed in parallel using the same reagents and protocol. As a control for both experiments, the subtraction was also carried out using human skeletal muscle mRNA that had been provided by the kit manufacturer.

First-Strand cDNA Synthesis

2 μg of PolyA⁺ RNA and 1 μl of cDNA synthesis primer (10 μM) were combined in a 0.5 ml Eppendorf tube and sterile H₂O was added where necessary to achieve a final volume of 5 μl. The contents were mixed gently and incubated in a thermal cycler at 70° C. for 2 min. The tubes were then cooled on ice for two minutes, after which 2 μl of 5×First-strand buffer, 1 μl of dNTP mix (10 mM each), sterile H₂O and 1 μl of AMV reverse transcriptase (20 units/μl) was also added. The tubes were then placed at 42° C. for 1.5 hr in an air incubator. First-strand cDNA synthesis was terminated by placing the tubes on ice. (the human skeletal muscle cDNA produced by this step was used as the ‘control driver’ in later steps).

Second-Strand cDNA Synthesis

48.4 μl of Sterile H₂O, 16.0 μl of 5×Second-strand buffer, 1.6 μl of dNTP mix (10 mM) and 4.0 μl of 20×second-strand enzyme cocktail were added to each of the first-strand synthesis reaction tubes. The contents were then mixed and incubated at 16° C. in a thermal cycler for 2 hr. 6 units (2 μl) of T4 DNA polymerase was then introduced and the tubes were incubated for a further 30 min at 16° C. In order to terminate second-strand synthesis, 4 μl of 20×EDTA/glycogen mix was added. A phenol:chloroform extraction was then carried out using the following protocol:

100 μl of phenol:chloroform:isoamyl alcohol (25:24:1) was added to the tubes which were then vortexed thoroughly and centrifuged at 14,000 rpm for 10 min at room temperature. The top aqueous layer was removed and placed in a fresh tube. 100 ill of chloroform:isoamyl alcohol (24:1) was then added to the aqueous layer and the tubes were again vortexed and centrifuged at 14,000 rpm for 10 min. 40 μl of 4 M NH₄OAc and 300 μl of 95% ethanol were then added and the tubes centrifuged at 14,000 rpm for 20 min. The supernatant was removed carefully, then 500 μl of 80% ethanol was added to the pellet. The tubes were centrifuged at 14,000 rpm for 10 min and the supernatant was removed so that the pellet could be air-dried. The precipitate was then dissolved in 50 μl of H₂O. 6 μl was transferred to a fresh microcentrifuge tube. The remainder of the sample was stored at −20° C. until needed.

Rsa I Digestion

All products of the above procedures were subjected to a restriction digest, using the restriction endonuclease Rsa I, in order to generate ds cDNA fragments that are short and thus are optimal for subtraction hybridisation due to the standard kinetics of the hybridisation. Also, as Rsa I makes a double stranded cut in the middle of a recognition sequence, ‘blunt ends’ of a known nucleotide sequence are produced allowing ligation of adaptors onto these ends in a later step. The following reagents were added to the 6 μl product of the second hybridisation (see above): 43.5 μl of ds cDNA, 5.0 μl 10×Rsa I restriction buffer and 1.5 μl of Rsa I (10 units/μl). The reaction mixture was incubated at 37° C. for 1.5 hr. 2.5 μl of 20×EDTA/glycogen mix was used to terminate the reaction. A phenol:chloroform extraction was then performed as above (second-strand cDNA synthesis section). The pellet produced was then dissolved in 5.5 μl of H₂O and stored at −20° C. until needed. The preparation of the experimental ‘Driver’ cDNAs and the control skeletal muscle cDNA was thus completed.

Adaptor Ligation

The adaptors were not ligated to the driver cDNA.

1 μl of each Rsa I-digested experimental cDNA (from the Rsa I Digestion above) was diluted with 5 μl of sterile water. Preparation of the control skeletal muscle tester cDNA was then undertaken by briefly centrifuging the tube containing control DNA (Hae III-digest of φX174 DNA [3 ng/μl]) and diluting 2 μl of the DNA with 38 μl of sterile water (to 150 ng/ml). 1 μl of control skeletal muscle cDNA (from the Rsa I Digestion) was then mixed with 5 μl of the diluted φX174/Hae III DNA (150 ng/ml) in order to produce the control skeletal muscle tester cDNA.

Preparation of the Adaptor-Ligated Tester cDNA

A ligation master mix was prepared by combining 3 μl of sterile water, 2 μl of 5×ligation buffer and 1 μl T4 DNA ligase (400 units/μl) per reaction. 2 μl of adaptor 1 (10 μM) was then added to 2 μl of the diluted tester cDNA. To this, 6 μl of the ligation master mix was also added. The tube was therefore labeled Tester 1-1. In a separate tube, 2 μl of the adaptor 2R (10 μM) was mixed with 2 μl of the diluted tester cDNA and 6 μl of the master mix. This tube was named Tester 1-2.

2 μl of Tester 1-1 and 2 μl of Tester 1-2 were then placed into fresh tubes. These would later be used as the unsubtracted tester control. The remainder of the contents of Tester 1-1 and Tester 1-2 tubes were then centrifuged briefly and incubated at 16° C. overnight. The ligation reaction was stopped by adding 1 μl of EDTA/glycogen mix and the samples were heated at 72° C. for 5 min in order to inactivate the ligase. In doing so, preparation of the experimental and control skeletal muscle adaptor-ligated tester cDNAs was complete.

1 μl from each unsubtracted tester control was then removed and diluted into 1 ml of water. These samples were set aside as they were to be used later for PCR (see below). All of the samples were stored at −20° C.

Analysis of Ligation Efficiency

1 μl of each ligated cDNA was diluted into 200 μl of water and the following reagents were then combined in four separate tubes:

Tube:

| Component | 1 | 2 | 3 | 4 |
|---|---|---|---|---|
| Tester 1-1 (ligated to Adaptor 1) | 1 | 1 | — | — |
| Tester 1-2 (ligated to Adaptor 2R) | — | — | 1 | 1 |
| G3PDH 3′ primer(10 μM) | 1 | 1 | 1 | 1 |
| G3PDH 5′ primer(10 μM) | — | 1 | — | 1 |
| PCR primer 1 (10 μM) | 1 | — | 1 | — |
| Total volume μl | 3 | 3 | 3 | 3 |

A master mix for all of the reaction tubes plus one additional tube was made up by adding 18.5 μl of sterile H₂O, 2.5 μl of 10×PCR reaction buffer, 0.5 μl of dNTP mix (10 mM), and 0.5 μl of 50×Advantage cDNA Polymerase Mix, per reaction, into a fresh tube. 22 μl of this master mix was then aliquotted into each of the 4 reaction tubes prepared above. The contents of the tubes were overlaid with 50 μl of mineral oil. The reaction mix was incubated in a thermal cycler at 75° C. for 5 min in order to extend the adaptors. The following protocol was then carried out immediately in a thermal cycler (Perkin-Elmer GeneAmp PCR Systems 2400): 94° C. for 30 sec (1 cycle), 94° C. 10 sec, 65° C. 30 sec and then 68° C. 2.5 min (25 cycles)

First Hybridisation

1.5 μl of the Adaptor 1-ligated Tester 1-1 was combined with 1.5 μl of the Rsa I-digested driver cDNA, prepared earlier and 1 μl of 4×Hybridisation buffer. This process was then repeated combining the Adaptor 2R-ligated Tester 1-2 with the Rsa I-digested driver cDNA and 4×hybridisation buffer. The samples were incubated in a thermal cycler at 98° C. for 1.5 min followed by incubation at 68° C. for 8 hr.

Second Hybridisation

1 μl of Driver cDNA (i.e. the Rsa I-digested cDNA (see above)), 1 μl 4×Hybridisation buffer and 2 μl Sterile H₂O were all combined in a fresh tube. 1 μl of this mix was then removed and placed in a new tube, overlaid with 1 drop of mineral oil and incubated at 98° C. for 1.5 min in order to denature the driver. The following procedure was used to simultaneously mix the driver with hybridisation samples 1 and 2 (prepared in the first hybridisation), thus ensuring that the two hybridisation samples were mixed together only in the presence of freshly denatured driver: A micropipettor was set at 15 pl. The pipette tip was then touched onto the mineral oil/sample interface of the tube containing hybridisation sample 2. The entire sample was drawn partway into the tip before it was removed from the tube in order to draw a small amount of air into the tip. The pipette tip was then touched onto the interface of the tube containing the freshly denatured driver (i.e. the tip contained both samples separated by a small pocket of air) before the entire mixture was transferred to the tube containing hybridisation sample 1. The reaction was then incubated at 68° C. overnight. 200 μl of dilution buffer was added to the tube, which was then heated in a thermal cycler at 68° C. for 7 min. The product of this second hybridisation was stored at −20° C.

PCR Amplification

Seven PCR reactions were set up: (1) The forward-subtracted experimental cDNA, (2) the unsubtracted tester control (see preparation of the adaptor ligated tester cDNA), (3) the reverse-subtracted experimental cDNA, (4) the unsubtracted tester control for the reverse subtraction, (5) the subtracted control skeletal muscle cDNA, (6) the unsubtracted tester control for the control subtraction, and (7) the PCR control subtracted cDNA (provided in the kit). The PCR control subtracted cDNA was required to provide a positive PCR control as it contained a successfully subtracted mixture of Hae III-digested φX174 DNA.

The PCR templates were prepared by aliquotting 1 μl of each diluted cDNA (i.e., each subtracted sample from the second hybridisation and the corresponding diluted unsubtracted tester control produced by the adaptor ligation, see above) into an appropriately labeled tube. 1 μl of the PCR control subtracted cDNA was placed into a fresh tube. A master mix for all of the primary PCR tubes, plus one additional reaction, was then prepared by combining 19.5 μl of sterile water, 2.5 μl of 10×PCR reaction buffer, 0.5 μl of dNTP Mix (10 mM), 1.0 μl of PCR primer 1 (10 μM) and 0.5 μl of 50×Advantage cDNA Polymerase Mix. 24 μl of Master Mix was then aliquotted into each of the 7 reaction tubes prepared above and the mixture was overlaid with 50 μl of mineral oil, before being incubated in a thermal cycler at 75° C. for 5 min in order to extend the adaptors. Thermal cycling was then immediately started using the following protocol: 94° C. 25 sec (1 cycle), 94° C. 10 sec, 66° C. 30 sec and 72° C. 1.5 min (32 cycles).

3 μl of each primary PCR mixture was then diluted in 27 μl of H₂O, 1 μl of each of these dilutions was then placed into a fresh tube.

A master mix for the secondary PCRs, (plus an additional reaction) was set up by combining 18.5 μl of sterile water, 2.5 μl of 10×PCR reaction buffer, 1.0 μl of Nested PCR primer 1 (10 μM), 1.0 μl of Nested PCR primer 2R (10 μM), 0.5 μl of dNTP Mix (10 mM) and 0.5 μl of 50×Advantage cDNA Polymerase Mix per reaction. 24 μl of this Master Mix was then added into each reaction tube containing the 1 μl diluted primary PCR mixture. The following PCR protocol was then carried out: 94° C. 10 sec, 68° C. 30 sec and 72° C. 1.5 min (12 cycles). The reaction products were then stored at −20° C.

Ligation into a Vector/Transformation & PCR

The products of the PCR amplification (enriched for differentially expressed cDNAs) were ligated into the pCR2.1-TOPO vector using a T/A cloning kit (Invitrogen), transformed into TOPO One Shot competent cells according to the manufacturers protocol and grown up on LB (Luria-Bertani) Agar plates overnight at 37° C. 1,000 colonies were then individually picked (using fresh sterile tips) and dipped into 5 μl of sterile water which had been aliquotted previously into 96 well PCR plates. The water/colonies were heated in a thermal cycler at 100° C. for 10 minutes in order to burst the cells, thus releasing the plasmids containing a differentially expressed cDNA insert. The 5 μl of water/plasmid was then used as a template in a PCR reaction (see below) using M13 Forward and Reverse primers (10 ng/μl), complementary to the M13 site present on either side of the cloning site on the vector. 5 μl of the PCR product was then run on a 2% agarose gel and stained by ethidium bromide. PCR products of an amplified insert were identified and 5 μl of the remainder of the PCR product (i.e. from the 15 μl that had not been run on the gel) was diluted 1/10 with water. 5 μl of the diluted PCR product was then used as a template in a sequencing reaction.

Sequencing

A sequencing reaction containing M13 primer (3.2 pmol/μl), ‘BigDye’ reaction mix (i.e. AmpliTaq® DNA polymerase, MgCl₂, buffer and fluorescent dNTPs [each of the four deoxynucleoside triphosphates is linked to a specific fluorescent donor dye which in turn is attached to a specific acceptor dye]) and cDNA template (diluted PCR product) was set up. The reaction was carried out on a thermal cycler for 25 cycles of 10 seconds at 96° C., 20 seconds at 50° C. and 4 minutes at 60° C. Each reaction product was then purified through a hydrated Centri-Sep column, and lyophilised. The pellets were resuspended in Template Supression Reagent and sequenced on an ABI Prism 310 Genetic Analyser. The analyser uses an ion laser to excite the specific donor dye that transfers its energy to the acceptor dye, which emits a specific energy spectrum that can be read by the sequencer.

The potentially upregulated genes of the streptozocin-induced diabetes experiment and of the CCI experiment were sequenced at Parke-Davis, Cambridge and at the applicant's core sequencing facility in Ann Arbor, Mich., USA.

Bioinformatics

The sequencing results were analysed using the computer program CHROMAS in which the vector and adaptor sequences were clipped off, leaving only the nucleotide sequence of the differentially expressed gene. Each sequence was then checked for homology to known genes, Expressed Sequence Tags (ESTs) and Proteins using various Basic Local Alignment Search Tool (BLAST) searches against the Genbank sequence database at the National Centre for Biotechnology Information, Bethesda, Md., USA (NCBI).

Lists were derived called STZup and STZdown and CCIup and CCIdown that contain the nucleic acid sequences from the forward and back subtracted libraries respectively. In each list there are given accession numbers and descriptions for the known rat genes identified, and where available corresponding mouse or human genes. Sequences that are considered to be of interest and that are up-regulated both in a streptozocin-induced diabetes model and in a chronic constrictive injury pain model are listed in Table 1 below.

References have been given where available for the sequences that have been found, and sequence listings have been given in the form in which they currently appear in publicly searchable databases e.g. the NCBI databaase (National Center for Biotechnology Information, National Library of Medicine, National Institutes of Health, Bethesda, Md., 20894, USA, www.ncbi.nlm.nih.gov). These sequence listings are given for the purposes of identification only. The invention includes the use of subsequently revised versions of the above sequences (which may incorporate small differences to the version set out herein) and homologous sequences or similar proteins in other species as determined by a high percentage identity (e.g. above 50%, preferably above 90%), length of alignment and functional equivalence.

| | Rat | Mouse | Human | | | Literature | |
|---|---|---|---|---|---|---|---|
| Expression | Accession | accession | Accession | | | Reference | |
| product or name | Number | number | Number | Reaction | Assay | No. | Seq. ID No's |
| A-raf oncogene, | X06942 | | U01337 | Mitogenic | Ser/Thr kinase | 1 | 1 Protein |
| liver expressed** | | | | signaling | | | 2. cDNA |
| Protein | D90164 | | X80910 | Cell signaling | Ser/Thr | 2 | 3. Protein |
| phosphatase 1 | | | | | phosphatase | | 4. cDNA |
| Phosphofructokin | U25651 | AF249894 | Y00698 | Glycolysis | Transferase | 3 | 5. Protein |
| ase, muscle | | | | | | | 6. cDNA |
| (PFK-M) | | | | | | | |
| Alkaline phospho- | D28560 | AF123542 | D45421 | Myelination | Phospho- | 4 | 7. Protein |
| diesterase 1* | | | | | diesterase | | 8. cDNA |
| Na + K + ATPase | M14512 | | | Membrane | Na+/K+ | 5 | 9. Protein |
| alpha + isoform | | | | potential | transport | | 10. cDNA |
| catalytic subunit | | | | | | | |
| Putative vacuolar | | U13837 | AF113129 | Ion channel | H+ transport | 6 | 11. Protein |
| ATP synthase | | | | | | | 12. cDNA |
| subunit A | | | | | | | |
| Hypoxia-inducible | AF057308 | AF003695 | U22431 | DNA binding | N/A | 7 | 13. Protein |
| factor-1 alpha | | | | | | | 14. cDNA |
| (Hifla) | | | | | | | |
| Cytochrome-c | M64496 | | | Energy | Oxidoreductase | 8 | 15. Protein |
| oxidse II, | | | | metabolism | | | 16. cDNA |
| mitochondrial | | | | | | | |
| Round spermatid | U97667 | | | Glutathione | Hydrolase | 9 | 17. Protein |
| protein RSP29 | | | | metabolism | | | |
| Putative 26S | | | Q16401 | Proteolysis | Traficking of | 10 | 18. Protein |
| proteasome | | | | | transmembrane | | |
| subunit S5B | | | | | proteins | | |
| Nap 1 | | | AB014509 | Binds to Nck | | 11 | 19. Protein |
| | | | | in signal | | | 20. cDNA |
| | | | | transduction; | | | |
| | | | | Nck is | | | |
| | | | | involved in | | | |
| | | | | cytoskeleton | | | |
| | | | | guidance | | | |
| Novel rat cofilin | | L29468 | AF134803 | Cytoskeleton | | 12 | 21. Protein |
| | | | | protein | | | 22. cDNA |
| Ganglioside | AB003575 | | O08765 | Similar to | | N/A | 23. Protein |
| expression factor- | BAA | | | human | | | (Human) |
| 2 (GEF-2) | 19975 | | | GABA(A) | | | 24. cDNA |
| | | | | receptor- | | | (mouse) |
| | | | | associated | | | |
| | | | | protein-like 2 | | | |
| | | | | and to mouse | | | |
| | | | | protein | | | |
| Putative KIAA | | | | Voltage- | | N/A | 25. (Protein) |
| 1117 | | | | gated | | | 26. cDNA |
| | | | | potassium- | | | |
| | | | | channel | | | |
| | | | | protein | | | |
| Putative F1-20 | | M83985 | | Synapse- | | 13 | 27. (Protein) |
| phosphoprotein | | | | specific | | | 28. cDNA |
| | | | | protein | | | |
| Proliferating cell | M24604 | | | DNA repair | | 14 | N/A (withdrawn) |
| nuclear antigen | | | | and | | | |
| (PCNA/cyclin) | | | | replication | | | |
| Ribosomal protein | J02650 | | | Protein | | 15 | 29. (Protein) |
| L19 | | | | synthesis | | | 30. cDNA |
| 14-3-3 protein | U37252 | D83037 | | Cell | | N/A | 31. (Protein) |
| zeta subtype | | | | signalling | | | 32. cDNA |
| 14-3-3 protein eta | D17445 | U57311 | X80536 | Signal | | 16 | 33. (Protein) |
| subtype | | | | transduction. | | | 34. cDNA |

### REFERENCES IN THE TABLE 1

1. Ishikawa, F., Takaku, F., Nagao, M. and Sugimura, T., The complete primary structure of the rat A-raf cDNA coding region: conservation of the putative regulatory regions present in rat c-raf, Oncogene Res., 1 (3), 243-253 (1987).

2. Sasaki, K., Shima, H., Kitagawa, Y., Irino, S., Sugimura, T. and Nagao, M., Identification of members of the protein phosphatase I gene family in the rat and enhanced expression of protein phosphatase 1 alpha gene in rat hepatocellular carcinomas, Jpn. J. Cancer Res. 81 (12), 1272-1280 (1990), Erratum: Jpn J Cancer Res July 1991; 82(7), 873.

3. Ma, Z., Ramanadham, S., Kempe, K., Hu, Z., Ladenson, J. and Turk, J., Characterization of expression of phosphofructokinase isoforms in isolated rat pancreatic islets and purified beta cells and cloning and expression of the rat phosphofructokinase-A isoform, Biochim. Biophys. Acta, 1308 (2), 151-163 (1996).

4. Narita, M., Goji, J., Nakamura, H. and Sano, K., Molecular cloning, expression, and localization of a brain-specific phosphodiesterase I/nucleotide pyrophosphatase (PD-I alpha) from rat brain, J. Biol. Chem., 269 (45), 28235-28242 (1994)

5. Shull, G. E., Greeb, J. and Lingrel, J. B., Molecular cloning of three distinct forms of the Na+, K+-ATPase alpha-subunit from rat brain, Biochemistry, 25, 8125-8132 (1986).

6. Laitala-Leinonen, T., Howell, M. L., Dean, G. E. and Vaananen, H. K., Resorption-cycle-dependent polarization of mRNAs for different subunits of V-ATPase in bone-resorbing osteoclasts, Mol. Biol. Cell 7 (1), 129-142 (1996).

7. Zou, A. P., Yang, Z. Z., Li, P. L. and Cowley A W, J. R., Oxygen-dependent expression of hypoxia-inducible factor-1alpha in renal medullary cells of rats, Physiol. Genomics (Online), 6 (3), 159-168 (2001).

8. Cao, J. L., Revzin, A. and Ferguson-Miller, S., Conversion of a mitochondrial gene for mammalian cytochrome c oxidase subunit II into its universal codon equivalent and expression in vivo and in vitro, Biochemistry, 30 (10), 2642-2650 (1991)

9. Ji, X., Moore, H. D., Russell, R. G. and Watts, D. J., cDNA cloning and characterization of a rat spermatogenesis-associated protein RSP29, Biochem. Biophys. Res. Commun., 241 (3), 714-719 (1997).

10. Deveraux, Q., Jensen, C. and Rechsteiner, M., Molecular cloning and expression of a 26 S protease subunit enriched in dileucine repeats, J. Biol. Chem. 270 (40), 23726-23729 (1995); Nomura, N., Nagase, T., Miyajima, N., Sazuka, T., Tanaka, A., Sato, S., Seki, N., Kawarabayasi, Y., Ishikawa, K. and Tabata, S., Prediction of the coding sequences of unidentified human genes. II., DNA Res. 1 (5), 223-229 (1994).

11. Suzuki, T., Nishiyama, K., Yamamoto, A., Inazawa, J., Iwaki, T., Yamada, T., Kanazawa, I. and Sakaki, Y., Molecular cloning of a novel apoptosis-related gene, human nap1 (NCKAP1), and its possible relation to alzheimer disease, Genomics, 63 (2), 246-254 (2000).

12. Ono, S., Minami, N., Abe, H. and Obinata, T., Characterization of a novel cofilin isoform which is predominantly expressed in mammalian skeletal muscle, J. Biol. Chem., 269, 15280-15286 (1994).

13. Lafer, E., Zhou, S., Sousa, R. and Tannery, N. H., Characterization of a novel synapse-specific protein. II. cDNA cloning and sequence analysis of the F1-20 protein, J. Neurosci., 12, 2144-2155 (1992)

14. Hsueh-Wel Chang et al., UV Inducubility of Rat Proliferating Cell Nuclear Antigen Gene Promoter, J. Cellular Biochem., 73, 423-432 (1999) and references cited therein.

15. Chan, Y.-L., Lin, A., McNally, J., Peleg, D., Meyuhas, O. and Wool, I. G., The primary structure of rat ribosomal protein L19: A determination from the sequence of nucleotides in a cDNA and from the sequence of amino acids in the protein, J. Biol. Chem., 262, 1111-1115 (1987)

16. Watanabe, M., Isobe, T., Okuyama, T., Ichimura, T., Kuwano, R., Takahashi, Y. and Kondo, H., Molecular cloning of cDNA to rat 14-3-3 eta chain polypeptide and the neuronal expression of the mRNA in the central nervous system, Brain Res. Mol. Brain Res., 10 (2), 151-158 (1991); Watanabe, M., Isobe, T., Ichimura, T., Kuwano, R., Takahashi, Y., Kondo, H. and Inoue, Y., Molecular cloning of rat cDNAs for the zeta and theta subtypes of 14-3-3 protein and differential distributions of their mRNAs in the brain, Brain Res. Mol. Brain Res., 25 (1-2), 113-121 (1994).

1. Use of:
(a) an isolated gene sequence that is up-regulated in the spinal cord of a mammal in response to mechanistically distinct first and second models of neuropathic or central sensitization pain;
(b) an isolated gene sequence comprising a nucleic acid sequence of Table 1;
(c) an isolated gene sequence having at least 80% sequence identity with a nucleic acid sequence of Table 1;
(d) an isolated nucleic acid sequence that is hybridizable to any of the gene sequences according to (a), (b) or (c) under stringent hybridisation conditions;
(e) a recombinant vector comprising a gene sequence or nucleic acid sequence according to any one of (a) to (d);
(f) a host cell comprising the vector according to (e);
(g) a non-human animal having in its genome an introduced gene sequence or nucleic acid sequence or a removed or down-regulated gene sequence or nucleic acid sequence according to any one of (a) to (d);
(h) an isolated polypeptide comprising an amino acid sequence at least 90% identical to an amino acid sequence encoded by a nucleotide sequence according to any one of (a) to (d), or a polypeptide variant thereof with sequential amino acid deletions from the C terminus and/or the N-terminus;
(i) an isolated polypeptide encoded by a nucleotide sequence according to any one of (a) to (d); or
(j) an isolated antibody that binds specifically to a polypeptide according to (h) or (i);
in the screening of compounds for the treatment of pain, or for the diagnosis of pain.

2. Use according to claim 1, wherein the isolated gene sequence is up-regulated both in response to streptozocin-induced diabetes and in response to surgical injury of a nerve leading to the spine.

3. Use according to claim 1 wherein the isolated gene sequence encodes a kinase.

4. Use according to claim 3, wherein the isolated gene sequence encodes an expression product or fragment thereof of phosphofructokinase, muscle (PFK-M) (U25651, AF249894, Y00698).

5. Use according to claim 1, wherein the isolated gene sequence encodes an expression product or fragment thereof of A-raf oncogene, liver expressed (X06942, U01337).

6. Use according to claim 1 wherein the isolated gene sequence encodes a phosphatase.

7. Use according to claim 1, wherein the isolated gene sequence encodes an expression product or fragment thereof of protein phosphatase I (D90164, X80910).

8. Use according to claim 1 wherein the isolated gene sequence encodes a phosphodiesterase.

9. Use according to claim 8, wherein the isolated gene sequence encodes an expression product or fragment thereof of alkaline phosphodiesterase I ((D28560, AF123542, D45421).

10. Use according to claim 1 wherein the isolated gene sequence encodes an ion channel protein.

11. Use according to claim 10, wherein the ion channel protein is putative vacuolar ATP synthase subunit A (U13837, AF113129) or Na⁺ K⁺ ATPase alpha+isoform catalytic subunit (M14512).

12. Use according to claim 1 wherein the isolated gene sequence encodes a DNA binding protein.

13. Use according to claim 12, wherein the isolated gene sequence encodes an expression product or fragment thereof of hypoxia-inducible factor-i alpha (Hif1a) (AF057308, AF003695, U22431).

14. Use according to claim 1 wherein the isolated gene sequence encodes an oxidoreductase or hydrolase.

15. Use according to claim 14, wherein the isolated gene sequence encodes an expression product or fragment thereof of cytochrome-c oxidase II, mitochondrial (M64496) or round spermatid protein RSP29 (U97667).

16. A non-human animal having in its genome an introduced gene sequence or a removed or down-regulated gene sequence, said sequence being up-regulated in the spinal cord of a mammal in response to first and second models of neuropathic or central sensitization pain.

17. A non-human animal according to claim 16, wherein said gene sequence becomes up regulated both in response to streptozocin induced diabetes and in response to chronic constriction injury.

18. A non-human animal according to claim 17, wherein the introduced gene sequence encodes a kinase, a phosphatase, a phosphodiesterase, an ion channel protein, a DNA binding protein, an oxidoreductase or a hydrolase.

19. A non-human animal according to claim 16 which is C. elegans.

20. A kit comprising;
(a) affinity peptide and/or ligand and/or substrate for an expression product of a gene sequence that is up-regulated in the spinal cord of a mammal in response to a mechanistically distinct first and second models of neuropathic or central sensitization pain; and
(b) a defined quantity of an expression product of a gene sequence that is up-regulated in the spinal cord of a mammal both in response to first and second models of neuropathic or central sensitization pain,
for simultaneous, separate or sequential use in detecting and/or quantifying up-regulation of a gene sequence in the spinal cord of a mammal in response to first and second models of neuropathic or central sensitization pain.

21. A kit according to claim 20, wherein the gene sequence encodes a kinase, a phosphatase, a phosphodiesterase, an ion channel protein, a DNA binding protein, an oxido reductase or a hydrolase.

22. A kit comprising:
(a) nucleic acid sequences capable of hybridization to a nucleic acid sequence that is up-regulated in the spinal cord of a mammal in response to first and second models of neuropathic or central sensitization pain; and
(b) a defined quantity of one or more nucleic acid sequences capable of hybridization to a nucleic acid sequence that is up-regulated in the spinal cord of a mammal in response to first and second models of neuropathic or central sensitization pain,
for simultaneous, separate or sequential use in detecting and/or quantifying up-regulation of a gene sequence in the spinal cord of a mammal in response to first and second models of neuropathic or central sensitization pain.

23. A kit of claim 22, wherein the gene sequence encodes a kinase, a phosphatase, a phosphodiesterase, an ion channel protein, a DNA binding protein, an oxido reductase or a hydrolase.

24. A compound that modulates the action of an expression product of a gene sequence that is up-regulated in the spinal cord of a mammal in response to first and second models of neuropathic or central sensitization pain.

25 A compound according to claim 24 wherein the gene sequence is listed in Table 1.

26. A compound according to claim 24 wherein the nucleotide sequence encodes a kinase, a phosphatase, a phosphodiesterase, an ion channel protein, a DNA binding protein, an oxidoreductase or a hydrolase.

27. A compound according to claim 24 for use as a medicament.

28. A compound according to claim 24 for the treatment or diagnosis of pain.

29. A pharmaceutical composition comprising a compound according to claim 24 and a pharmaceutically acceptable carrier or diluent.

30. Use of a compound according to claim 24 in the manufacture of a medicament for the treatment or diagnosis of pain.

31. Use of a compound according to claim 24 in the manufacture of a medicament for the treatment or diagnosis of chronic pain.

32. A method of treatment of pain, which comprises administering to a patient an effective amount of a compound according to claim 24.


SEQUENCE LISTING (34 sequences)

SEQ ID NO: 1 - protein, 604 aa, Rattus norvegicus
  A-raf protein (AA 1-604)
MEPPRGPPASGAEPSRAVGTVKVYLPNKQRTVVTVRDGMSVYDSLDKALKVRGLNQDCCVVYRLIKGRKTVTAWDTAIAPLDGEELIVEVLEDVPLTMHNFVRKTFFSLAFCDFCLKFLFHGFRCQTCGYKFHQHCSSKVPTVCVDMSTNRRQFYHSIQDLSGGSRQQEVPSNLSVNELLTPQGPSPFTQQRDQEHFSFPAPANPPLQRIRSTSTPNVHMVSTTAPMDSSLMQFTAQSFSTDAAGRGGDGAPRGSPSPASVSSGRKSPHSKLPAEQRERKSLADEKKKVKNLGYRDSGYYWEVPPSEVQLLKRIGTGSFGTVFRGRWHGDVAVKVLKVAQPTAEQAQAFKNEMQVLRKTRHVNILLFMGFMTRPGFAIITQWCEGSSLYHHLHVADTRFDMVQLIDVARQTAQGMDYLHAKNIIHRDLKSNNIFLHEGLTVKIGDFGLATVKTRWSGAQPLEQPSGSVLWMAAEVIRMQDPNPYSFQSDVYAYGVVLYELMTGSLPYSHIGSRDQIIFMVGRGYLSPDLSKIFSNCPKAMRRLLTDCLKFQREERPLFPQILATIELLQRSLPKIERSASEPSLHRTQADELPACLLSAARLVP

SEQ ID NO: 2 - DNA, 2288 bp, Rattus norvegicus
  cDNA coding region for A-raf protein
gcggtagcgtgtgacaggaggcctatggcacctgcccagtcctacctcagcccatcttgaaaaaatccttagacaacatggaaccaccacgaggccctcctgctagtggggctgagccatctcgggcagttggcactgtcaaagtgtacctgcctaacaagcaacgcacagtggtgactgtccgggatggcatgagtgtctatgactctttggacaaggccctcaaggtgcggggtctcaatcaggactgctgtgtggtctacagactcatcaaaggaagaaagacagtcactgcctgggacacagccattgcacctctggatggcgaggagctcattgtggaggtcctggaagatgtgccactgaccatgcacaattttgtacggaagacattcttcagtttggccttctgtgacttctgccttaagtttctgttccacggctttcgctgccaaacctgtggctacaagttccaccagcattgttcctccaaggtccccacggtctgcgttgacatgagtaccaaccgccgacagttctaccacagcatccaggatttgtctggaggctccaggcagcaggaggttccctcaaatctctctgtgaatgagctgctaaccccccagggtcccagtccctttacccagcaacgtgaccaggagcacttctccttccctgcccctgccaatcccccactgcagcgcatccgctccacatctactcctaacgtccacatggtcagcaccacagctcccatggactccagcctcatgcagtttactgctcagagcttcagcaccgatgctgctggtagaggtggtgatggagctcctcggggtagccctagcccagctagtgtgtcttcagggaggaagtccccacattccaagttacctgcagaacagcgggaacggaagtccttggcagatgaaaagaaaaaagtgaagaacctggggtaccgggactcaggctattactgggaggtgccacccagtgaggtacagctgttgaagaggatcgggacaggctcttttggcactgtgtttcgggggcgttggcatggcgatgtagctgtgaaagtgctcaaggtggcccaacctaccgctgagcaggcccaggccttcaagaatgagatgcaggtgctcaggaagacacgacatgtcaacattttgctgtttatgggtttcatgactcggccagggtttgccatcatcacacagtggtgtgagggttccagcctctaccaccacctacatgtggctgacacgcgctttgacatggtccagctcattgatgtggcccggcagactgcccagggcatggactacctccacgccaagaacatcattcaccgagacctaaagtccaacaatatcttcctacatgaggggctcacagtcaagattggtgacttcggcctggccacagtgaagacacgatggagtggggcccagcccttagagcagccctcagggtctgtgctgtggatggcagctgaggtgatccgaatgcaggacccgaacccctacagcttccagtcggatgtctatgcctatggtgttgtgctctatgagcttatgaccggctcactgccctacagccacattggcagccgtgaccagatcatctttatggtgggtcgtggctatctgtctccggacctcagcaaaatcttcagtaattgccccaaagccatgaggcgcttgctgactgactgcctcaagttccagcgggaggagcggcctctatttccccagattctggccacgatcgagctgctgcagcggtcactccccaagattgagcggagtgcctccgaaccctccttgcaccgtacccaggctgatgagttgcctgcctgccttctcagcgcagcccgccttgtgccttagactccactcccagcccactagggagccattttcagcttaccatgccaaggcacccccttcctaccagccaatcattgttctgtctgtgccctgatactgcctcaggattccctatcccacaccctgggaaatttgggggactccaaaaactgaggtcccctgcttcctccataatttggtctcctcttggctttggggatagttctaatttggagagctgttttacctccaatggctgggattcagtgcaaagattccactcggaacctctttataaagttttcgcctgacatgtcttcactgaattatggggttcccagcaccccatgcggatttgggagtttccctttgtctccccccactattcaaggactctcctctttaccaagaagcaca

SEQ ID NO: 3 - protein, 327 aa, Rattus norvegicus
  Protein phosphatase 1, catalytic subunit
MADGELNVDSLITRLLEVRGCRPGKIVQMTEAEVRGLCIKSREIFLSQPILLELEAPLKICGDIHGQYTDLLRLFEYGGFPPEANYLFLGDYVDRGKQSLETICLLLAYKIKYPENFFLLRGNHECASINRIYGFYDECKRRFNIKLWKTFTDCFNCLPIAAIVDEKIFCCHGGLSPDLQSMEQIRRIMRPTDVPDTGLLCDLLWSDPDKDVQGWGENDRGVSFTFGADVVSKFLNRHDLDLICRAHQVVEDGYEFFAKRQLVTLFSAPNYCGEFDNAGGMMSVDETLMCSFQILKPSEKKAKYQYGGLNSGRPVTPPRTANPPKKR

SEQ ID NO: 4 - DNA, 2706 bp, Rattus norvegicus
  cDNA phosphatase 1, catalytic subunit
cgcccttgttcccgctgcggggaggagagtctggtgcctacaagatggcggacggggagctgaacgtggacagcctcatcacccgcctgctggaggtacgaggatgtcgtccgggaaaaattgtgcagatgactgaagcagaagtccgaggactgtgtatcaagtctcgtgaaatctttcttagccagcctattcttttggaattggaagcgccactgaagatttgtggagatattcatggacagtatacagacttactgagattatttgaatatggaggttttccaccagaagccaactatcttttcttaggagattatgtggacagaggaaagcagtctttggaaaccatctgtttgctattggcttacaaaatcaaatacccagagaacttctttcttctacgaggaaaccatgagtgtgctagcatcaaccgcatttatggattctatgatgagtgcaaacgaagatttaatattaaattgtggaagacattcactgattgttttaattgtctgcctatagctgctattgttgatgagaaaatcttctgctgtcatggaggactgtcaccagacctacagtctatggaacagattcggagaattatgagacccactgacgtacctgatacaggtttgctttgtgatttactgtggtccgacccagataaggatgtacaaggctggggagaaaatgatcgtggtgtttcttttacttttggagctgatgtagtcagtaaatttctgaatcgtcatgatttggacttgatttgtcgagcccatcaggtggtagaagatggatatgaattttttgctaaacgacaattggtaactttattttctgccccaaattactgcggcgagtttgacaatgctggtggtatgatgagtgtggatgaaactttgatgtgttcattccagatattgaaaccatctgaaaagaaagctaagtaccagtatggtgggctgaattctggacgtcctgtcactccgcctcgaacagctaatccaccgaagaaaaggtgaagacaggaattccggaaagagaaaccatcagatttgttaaggacatacttcataatatataagtgtgcactgtaaaaccatccagccattcgacaccctttatgatgtcacacctttaacttaaggagacgggtaaaggatcttaaatttttttctaatagaaagatgtgctacactgtattgtaataagtatactctgttataatattcaacaaagttaaatccaaattcaaaagtatccattaaagttctatcttctcatatcacagtttttaaagttgaaagcatcccagttaaactagctgcgttagttacccagatgagagcatgaagatccatctgtgtaatgtggctttagtgttgcttggttgtttctttattttgggcttgttttgttttgtttgtttttgctagaataatggcatctacttttcctatttttccctaaccattttaaaaagtgaaaatgggaagagctttaaagacattcaccaactattcttttccttcacttatctacttaaggaactgttggatcttactaagaaaacttacgcctcataataaaaaggaactttagaggccgataggttttaaaaatatacaaactatttgatccaatgattttaatcaaacagtttgactgggcaaactttgcagctgataatgactatttcgctttttacaaattgccactgatttggatttgtgcactctaacctttaatttattgatgctctattgtgcagtagcatttcatttaagataaggctcatatagtactatccaaaattagttggtaatgtgattatgtggtaccttggctttaggttttaattcgcacgaaacaccttttggcatgcttaactttctggtattatcctcacctgcattggttttgttttttggggtttttgttgttgtttgtttgtttgtttttagatccacagaacatgagaatcctttttgacaagccttggaaagctggctcttctttccctctctatgtgaaggatgtatttaaatgaacactggtcagtgggacattgtcagctctgagtattgggtgcttcactgtctaataattgccatgtgaatgttgtttttgactgtaaggctatgtcactaaagatttttactctgcgttttcataatcaaaggtcatgatgtgtatagacatgctttgtagtgaagtatagtagcaataatttctgcacatgatcaagagtttattgcagcatttctttccctgttctctcttttttaagggttagcattaacaaatgtcaaggaatagcaaagtcaacaaagactttaggaggtggaattaagaacacacagatttgtgatctttggatgtgacacttattggatgttattctaaagtcttattgaacattgtcaaatttgtaagcttcatggggatggacataatgtttatataatgcccttcttatgtgttaccatagatgtgaaaccttatattgtctttgaaaatgttaaattgagaactctgttaacattttatggattggcacattatattactgcaagaaacatttgattttcagcacagtgcaaaagttctttaaaatgcatatgtctttttttctaattcaattttgtttaaagcacattttaaatgtagttttctcatttagtaaaaagttgtctaatt

SEQ ID NO: 5 - protein, 776 aa, Rattus norvegicus
  Protein phosphofructokinase muscle isozyme
MTHEEHHEAKTLGIGKAIAVLTSGGDAQGMNATVRAVVRVGIFTGLRVFFVHEGYQGLVDGGEHIREATWESVSMMLQLGGTVIGSARCKDFREREGRLRAAHNLVKRGITNLCVIGGDGSLTGADTFRSEWSDLLNDLQKDGKITAEERTKSSYLNIVFLVGSIDNDFCGTDMTIGTDSALHRIVEIVDAITTTAQSHQRTFVLEVMGRHCGYLALVTSLSCGADWVFIPECPPDDDWEEHLCRRLSETRTRGSRLNIIIVAEGAIDKNGKPITSEDIKNLVVKRLGYDTRVTVLGHVQRGGTPSAFDRILGSRMGVEAVMALLEGTPDTPACVVSLSGNTAVRLPLMECVQVTKDVTKAMDEKRFDEAIKLRGRSFMNNWEVYKLLAHVRPPVSKGGLHTVAVMNVGAPAAGMNAAVRSTVRIGLIQGNRVLVVHDGFEGLAKGQIEEAGWSYVGGWTGQGGSKLGTKRTLPKKNLEQISANITKYNIQGLVIIGGFEAYTGGLELMEGRKQFDELCIPFVVIPATVSNNVPGSDFSIGADTALNTICTTCDRIKQSAAGTKRRVFIIETMGGYCGYLATMAGLAAGADAAYIFEEPFTIRDLQVNVEHLVQKMKTTVKRGLVLRNEKCNENYTTDFIFNLYSEEGKGIFDSRKNVLGHMQQGGNPTPFDRNFATKMGAKATNWMSGKIKESYRNGRIFANTPDSGCVLGMRKRALVFQPVTELKDQTDFEHRIPKEQWWLKLRPILKILAKYEIDLDTSDHAHLEHISRKRSG

SEQ ID NO: 6 - DNA, 2757 bp, Rattus norvegicus
  cDNA phosphofructokinase muscle isozyme
gccagcgaggagagctaaaactacaagagtggatcatgacccatgaagagcatcatgaagccaaaaccctggggatcggcaaggccatcgccgtgttgacctctggtggagatgcccaaggtatgaatgctacggtcagggctgtggtacgagttggcatcttcaccgggctccgcgtcttctttgtccatgagggttaccaaggcctggtggatggtggagagcacatcagggaggccacctgggagagcgtgtccatgatgctccagctgggaggcacggtgattggaagtgcccgatgcaaggacttccgggagcgagaaggacgactccgagctgcccacaacctggtgaagcgggggatcaccaatctgtgtgtcatcggaggcgatggcagcctcactggggctgacacttttcgttcagagtggagtgacttattgaatgacctccagaaagatgggaagatcacagccgaggagcgtacaaagtccagctacctgaacatcgttttcctggttggctcaatcgacaatgacttctgtggcactgatatgaccattggtactgactctgccctgcaccgcattgtagagatcgtggacgccatcaccaccaccgctcagagccaccagaggacatttgtgttagaagtgatgggccgccactgtggatacctggcccttgtcacctctctgtcgtgtggggccgactgggttttcattcccgagtgtccgccagatgacgactgggaagaacacctttgtcgccgtctcagtgagacaagaacccgtggttctcgtctcaacatcatcattgttgctgagggtgcaatcgacaaaaacgggaagccaatcacctcagaagacatcaagaacctggtggtaaagcgtcttggatatgataccagggtcactgttctgggacatgtacagaggggtgggacaccatcagcctttgaccggatcctgggcagcaggatgggtgtggaagcagtgatggcacttttggaggggaccccagacaccccagcctgtgtggtgagcctctctggtaatacggctgtgcgcctgcccctcatggagtgtgtccaggtgaccaaagacgtgaccaaggctatggatgagaagagatttgatgaagccattaagctgagaggccggagcttcatgaacaactgggaggtatacaagcttctagctcatgtcagacccccagtctctaagggcgggttgcacacggtggctgtgatgaacgtgggggccccagctgctggaatgaatgctgcagttcgctctactgtgaggattggccttatccaaggcaaccgagtgctggtcgtgcatgatggctttgagggtctggccaaaggtcagattgaggaagctggctggagctatgttggaggctggactggtcaaggtggttccaaacttggtactaaaaggactctacccaagaagaacctggaacagatcagtgccaacataaccaagtataacatccagggcctggttatcattgggggctttgaggcttacacagggggcttggagctgatggagggcaggaagcagtttgatgagctctgcatcccatttgtggtcattcccgccacggtttccaataatgtcccagggtcagacttcagcatcggggctgacacagcactgaacaccatctgcacgacctgtgaccgaatcaagcagtctgcagcaggcaccaagcggcgagtgtttatcatcgagacgatgggtggttactgtggctatctggccaccatggcaggactggcggctggggctgatgctgcctacatttttgaggagcccttcaccatccgagatctccaggttaatgttgaacatctggtgcagaagatgaaaacaactgtgaagagaggcctggtgctgaggaatgagaagtgcaacgagaactacactactgatttcattttcaacctgtactctgaggaggggaagggcatcttcgacagcaggaagaacgtgcttggccacatgcagcagggtggaaacccaactccctttgacaggaattttgccaccaagatgggtgctaaggctacgaattggatgtctgggaaaatcaaagagagttaccgtaatggacggatctttgccaacactcccgactcaggttgtgttctggggatgcgtaagagggccctggtctttcagccagtgactgagctgaaggaccagacagattttgagcaccgaatccccaaagaacagtggtggctgaagctgaggccaatcctcaaaatcctggccaagtacgagattgatctggacacctctgaccacgcccacctggagcacatttccaggaagcggtctggagaagctgctgtctagaccactttggagtgaggggaaaatcacctgatcatggtcagctcacaccctggtagacctcagcccatggcttctcagtgttgtagcccagcccctacccttctaggtttccctgtactctgtacctgcaaccaggatcactgtggccaggtgttgggggaggggtggtgagtgcctctcctaggtagctgtccttccttgcaccctggcttcatctgtcacacaggctggatgtctctagtgctactgctagatttcagcttctcaagagtaaaagtgagctttatttatttctttgtgataacaaagagtcctaggtcctttccttgtaccacagtgaagtgtaactacactaataaaagccagctggccactgtga

SEQ ID NO: 7 - protein, 885 aa, Rattus norvegicus
  Protein Phosphodiesterase 1
MARQGCLGSFQVISLFTFAISVNICLGFTASRIKRAEWDEGPPTVLSDSPWTNTSGSCKGRCFELQEVGPPDCRCDNLCKSYSSCCHDFDELCLKTVRGWECTKDRSGEVRNEENACHCPEDCLSRGDCCTNYQVVCKGESHWVDDAARNQSSECLQVCPPPLIIFSVDGFRASYMKKGSKVMPNIEKLRSCGTHVPYTRPVYPTKTFPNLYTLATGLYPESHGIVGNSMYDPVFDASFHLRGREKFNHRWWGGQPLWITATKQGVRAGTFFWSVSIPHERRILTILQWLSLPDNERPSVYAFYSEQPDFSGHKYGPFGPEMTNPLREIDKTVGQLMDGLKQLRLHRCVNVIFVGDHGMEDVTCDRTEFLSNYLTNVDDITLVPGTLGRIRAKSINNSKYDPKTIIANLTCKKPDQHFKPYMKQHLPKRLHYANNRRIEDIHLLVDRRWHVARKPLDVYKKPSGKCFFQGDHGFDNKVNSMQTVFVGYGPTFKYRTKVPPFENIELYNVMCDLLGLKPAPNNGTHGSLNHLLRTNTFRPTMPDEVSRPNYPGIMYLQSEFDLGCTCDDKVEPKNKLEELNKRLHTKGSTEAETGKFRGSKHENKKNLNGSVEPRKERHLLYGRPAVLYRTSYDILYHTDFESGYSEIFLMPLWTSYTISKQAEVSSIPEHLTNCVRPDVRVSPGFSQNCLAYKNDKQMSYGFLFPPYLSSSPEAKYDAFLVTNMVPMYPAFKRVWAYFQRVLVKKYASERNGVNVISGPIFDYNYDGLRDTEDEIKQYVEGSSIPVPTHYYSIITSCLDFTQPADKCDGPLSVSSFILPHRPDNDESCNSSEDESKWVEELMKMHTARVRDIEHLTGLDFYRKTSRSYSEILTLKTYLHTYESEI

SEQ ID NO: 8 - DNA, 3216 bp, Rattus norvegicus
  cDNA Phosphodiesterase 1
ggtacccaacagcctgaactcagagccccgagagcagagcattcagggcaagcagaaacaccctgcagaggctttccaagaatccctcggcatggcaagacaaggctgtctcgggtcattccaggtaatatccttgttcacttttgccatcagtgtcaatatctgcttaggattcacagcaagtcgaattaagagggcagaatgggatgaaggacctcccacagtgctgtctgactctccatggaccaacacctctggatcctgcaaaggtagatgctttgagcttcaagaggttggccctccagactgtcggtgtgacaacctgtgtaagagctacagcagctgctgccacgatttcgatgagctctgtttgaaaacagtccgaggctgggagtgcaccaaagacagaagtggggaagtacgaaacgaggaaaatgcctgtcactgcccagaagactgcttgtccaggggagactgctgtaccaactaccaagtggtctgcaaaggagaatcacactgggtagatgatgctgcgagaaatcaaagttccgaatgcctgcaggtttgtccgcctccgttaatcatcttctctgtggatggtttccgtgcatcatacatgaagaaaggcagcaaggttatgcccaacattgagaaactgcggtcctgtggcacccatgtcccctacacgaggcctgtgtaccccacaaaaaccttccctaatctatatacgctggccactggtttatatccggaatcccatggaattgtcggtaattcaatgtatgatcctgtctttgatgcttcgttccatctacgagggcgagagaagtttaatcataggtggtggggaggccaaccgctatggattacagccaccaagcaaggggtgagagctggaacattcttttggtctgtgagcatccctcatgaacggaggatcctaaccattcttcagtggctttctctgccagacaacgagaggccttcagtttatgccttctactcagagcagcctgatttttctggacacaagtacggcccttttggccctgagatgacaaatcctctgagggagattgacaagaccgtggggcagttaatggatggactgaaacaactcaggctgcatcgctgtgtgaacgttatctttgttggagaccatggaatggaagatgtgacatgtgacagaactgagttcttgagcaactatctgactaatgtggatgacattactttagtgcctggaactctgggaagaattcgagccaaatctatcaataattctaaatatgaccctaaaaccattattgctaacctcacgtgcaaaaaaccggatcagcactttaagccttacatgaaacagcaccttcccaaacggttgcactatgccaacaacagaagaattgaagacatccatttattggtcgatcgaagatggcatgttgcaaggaaacctttggacgtttataagaaaccatcaggaaaatgttttttccagggtgaccacggctttgataacaaggtcaatagcatgcagactgttttcgtaggttatggcccaacttttaagtacaggactaaagtgcctccatttgaaaacattgaactttacaatgttatgtgcgatctcctaggcttgaagcccgctcccaataatggaactcatggaagcttgaatcacctactgcgtacaaatacctttaggccaaccatgccagacgaagtcagccgacctaactacccagggattatgtaccttcagtccgagtttgacctgggctgcacctgtgacgataaggtagagccaaagaacaaattggaagaactcaataaacgtcttcataccaaaggatcaacagaagctgaaaccgggaaattcagaggcagcaaacatgaaaacaagaaaaaccttaatggaagtgttgaacctagaaaagagagacatctcctgtatggacggcctgcagtgctctatcggactagctatgatatcttataccatacggactttgaaagtggttatagtgaaatattcttaatgcctctctggacatcgtataccatttctaagcaggctgaggtctccagcatcccagaacacctgaccaactgtgttcgtcctgatgtccgtgtgtctccaggattcagtcagaactgtttagcttataaaaatgataaacagatgtcatatggattcctttttcctccctacctgagctcctccccagaagctaagtatgatgcattcctcgtaaccaacatggttccaatgtaccccgccttcaaacgtgtttgggcttatttccaaagggttttggtgaagaaatatgcttcagaaaggaatggagtcaacgtaataagtggaccgatttttgactacaattacgatggcctacgtgacactgaagatgaaattaaacagtatgtggaaggcagctctatacctgtccccacccactactacagcatcatcaccagctgcctggacttcactcagcctgcagacaagtgtgacggtcccctctctgtgtcttccttcatccttcctcaccgacccgacaatgatgagagctgtaatagctccgaggatgagtcgaagtgggtagaggaactcatgaagatgcacacagctcgggtgcgggacattgagcacctcactggtctggatttctaccggaagactagccgtagctattcggaaattctgaccctcaagacatacctgcatacatatgagagcgagatttaactttctgggcctgggcagtgtagtcttagcaactggtgtatatttttatattgtgtttgtatttattaatttgaaccaggacacaaacaaacaaagaaacaaacaaataaaaaaaaaaaccacttagtattttaatcctgtaccaaatctgacatattaagctgaatgactgtgctattttttttccttaattcttgatttagacagagttgtgttctgagcagagtttatagtgaacactgaggctcacaatccaagtagaagctacgtggatctacaaggtgctgcaggttgaaaatttgcattgaggaaatattagttttccagggcacagtcaccacgtgtagttctgttctgttttgaaagactgattttgtaaaggtgcattcatctgctgttaactttgacagacatatttatgccttatagaccaagcttaaatataataaatcacacattcagatttt

SEQ ID NO: 9 - protein, 1020 aa, Rattus norvegicus
  Protein Na+, K+ ATPase alpha(+) isoform catalytic subunit
MGRGAGREYSPAATTAENGGGKKKQKEKELDELKKEVAMDDHKLSLDELGRKYQVDLSKGLTNQRAQDILARDGPNALTPPPTTPEWVKFCRQLFGGFSILLWIGALLCFLAYGILAAMEDEPSNDNLYLGIVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNICFFSTNCVEGTARGIVIATGDRTVMGRIATLASGLEVGQTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPVSQVNPREAKACVVHGSDLKDMTSEQLDEILRDHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTTNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 10 - DNA, 5109 bp, Rattus norvegicus
  cDNA Na+, K+ ATPase alpha(+) isoform catalytic subunit
tctgccagggtctccagctgccccagacaggcggtgtggtcttgggatcctcctggtgacctttccagcctaggtcccctcagccactctgccccaagatgggacgtggggcagggcgtgagtactcgcctgccgccaccactgcggagaatgggggtggcaagaagaaacagaaagagaaggagctcgatgagctgaagaaggaggttgccatggatgaccacaagctgtccttggatgagctgggccgaaaataccaagtggatctgtccaagggcctcaccaaccagcgagctcaggatattctggctagagacggacccaacgccctcactccaccccctacaactcctgagtgggtcaagttctgccgtcagctttttgggggcttctctatcctgctgtggattggggcgcttctctgcttcttagcctatggtatcctggccgccatggaggacgaaccatccaatgacaatttatatctaggtatcgtgctagcagctgtagttatcgtcactggctgcttctcctactaccaggaagccaaaagctccaagattatggactccttcaagaacatggtgcctcagcaagctctggtcatccgagagggagagaagatgcagatcaatgcagaggaggtggtcgtgggagacctggtggaagtgaagggtggagaccgtgtccccgctgacctccggatcatctcctcccacggttgcaaggtggataactcatccctgacaggggagtcggagccccagacccggtcccctgagttcacccatgagaatcccttggagacccgcaatatctgtttcttctctaccaactgtgtggaaggcactgccaggggcattgtgatcgccacaggtgaccggacggtgatgggccgcatagccactcttgcctctggcctagaggtgggacagacgccgatagccatggagatcgagcatttcatccagctgatcacgggggtggccgtgttcctgggggtctccttctttgttctgtcgctcatcctgggctacagctggctggaggctgtcatcttcctcatcggcatcatcgtagccaacgtccccgaagggctcttggccactgttactgtgtgcctgacgctgacagccaagcgcatggctcgcaagaactgcctggtgaagaacctggaggcggtggagacgctgggctccacgtccaccatctgctcggacaagacaggcaccctcacccagaaccgcatgacggtggctcacatgtggtttgacaaccagatccatgaggctgacaccactgaagatcagtctggggccacttttgacaagcggtccccgacgtggacagccctgtctcggatcgctggtctctgcaatcgtgccgtcttcaaggctgggcaggagaacatctccgtgtctaagcgggacacagctggtgacgcctctgagtcagctctgctcaagtgcatcgagttgtcctgtggctcagtgaggaagatgagggacaggaatcccaaggtggcagaaattcccttcaactctaccaacaaatatcagctttccatccatgagagggaagacagcccccagagccatgtgctggtgatgaaaggtgccccggagcgcatcctggaccgatgctctaccatcctggtacagggcaaggagatccctcttgacaaggagatgcaagatgcctttcaaaacgcctacatggagctgggaggactcggggagcgagtgctgggcttctgtcagctgaacctgccttctggaaagtttcctcggggcttcaaatttgacacggatgagctgaactttcccacagagaagctctgctttgtggggctcatgtctatgattgatccccccagagcagctgtgccagatgctgtgggcaagtgcagaagtgcaggcatcaaggtgatcatggtgactggggatcaccctatcacagccaaggccattgccaaaggtgtgggcatcatatcagagggtaacgagactgtggaagacattgcagccaggctcaacattcctgtgagtcaagtcaatcccagagaagccaaggcatgtgtagtgcacggctcagacctgaaggacatgacttcagagcagctggatgagatcctcagggaccacacggagatcgtgtttgcccggacctcccctcagcagaagctcatcattgtggagggctgtcagaggcagggagccatcgtggcagtgactggtgacggggtgaacgactcccccgcgctgaagaaggctgacattggcattgccatgggcatctctggctctgatgtctctaagcaggcagctgacatgatccttctcgacgacaactttgcctccattgtgacgggcgtggaggaggggcgcctgatctttgacaacctgaagaagtccatcgcgtacaccctgaccagcaacatccctgagatcacccccttcctgctgttcatcattgccaacatcccccttcccctgggcaccgtgaccatcctgtgcatcgacctgggcacagacatggttcctgccatctcattagcatacgaagcggctgagagcgacatcatgaagaggcagccacggaactcccagacggacaagctggtgaacgagaggcttatcagcatggcttacggacagatcggcatgatccaggctctgggcggcttcttcacctactttgtaatactggcagagaacggcttcctgccatcgaggctgcttgggatccgccttgactgggatgatcggactaccaacgacctggaggacagctatggacaagagtggacctatgagcagcggaaggtggtggagttcacatgccacacggccttctttgccagcatcgtggttgtgcagtgggctgacctcatcatttgcaagacccggcgcaactcggtgttccagcagggcatgaagaacaagatcctgatttttgggctgctagaagagacggctctggctgccttcctgtcttactgcccgggtatgggggtggccctccgaatgtacccactcaaggtcacgtggtggttctgtgcctttccctacagtctcctcatcttcatctatgatgaagtccgaaagctcatcctgcggcggtaccctgggggctgggtggagaaggagacgtactactgagctcaccgacaaaaggaagaacaggggagatggggtgctccagaggggctggtgggtgttgtggtgaagggaagggctggggagacacaaggaagcgatggtggcgtgaactcagtgggtaggcttgggtaaataaacttgaggagactgctccaactgctccataggtcccgctgtgaaccctaagacagtgcatgttggggtcgcctcctcagatccttcccgtcccactctcccacattgtctacattttctgaagaaccgggggtcgccctagccctccctgtgtcccagtccttcaccctcacctgcattattccattattcaaacagatcaacacccaaaggttaatcctgtctaaccctggaggaaagcctgtcagaccaccagtacccaccactaccaccaccaccaccaccaccaccaccaccaccaccaccaccaccaccaccccctgttcactcctcttcccattcttgccttcctcaccttcctgcctgagtcttcccttgctcctccccttacaccttgaaaacacaaaattctgcttctgtgagtgcaagagcctagggccagaaaaggaagccagttggagagatggggcctgtctcccagccaagaccagtcaggaaccagaggggagctgggctggcaagtggaggttggggtgcactggctgagaaagaaagaaaggaaggaaggaaggaaggaaggaaggaaggaagaaaggaaggaaggaagtgaccacaggtgtgtcatctccagccttcaggtatatgggacaggctctggatctgtgagagacttaagagactagcacaccagcagaccaaattcccatctcatcagactagcagtaagtgccacccagtgcccctctgacccttgggtagtggtcctctctgtccacaaggctcagatttcacagaaggttcagctatctcaaccacatactcttgggaacaccccccttctttagaataattagttctctggggcctcgtgctgttctgagagccccattagctgccacttctcctcgtgctctctcactgccttctgcttcctacccacctgctgaacccacgttatgtccagtatcgccttgcttgtcctgaaaaaggatctcttggccattggcaggaatcagtgtagaaatgtttccaggacatccctgactttcgggaacatgcagaatcagtgtagctcatgacacagtcagaaactttagacacaagagaaattcttaagagacctatgcacctttgacctctcagattgagacaggaagctggcttcaggttcctatggcatagagttatctttccttactctcttctcaacctaacgtattcgctcttcagacagctgcctgttctctaatcctggcctagaaagcatagcatagatgcacctggatcaatgagggaagcaagagagaatcagcaaggaaactggaaggcttgaggtgggaatatgaaagtcaagacaagcatcaagccaggcccaagggcctcccaaaggctactgtctcatcgtggtggatggagttttgccttaccctaaataccttgaaatttgtcagtcatgcatagccttcagtcggaagtcaaaatgggacaccgtatttatttgggcctgactaatttgagatcactgactttgaacaaaagtttacctttgcacaatcaataaaatcatctgctaggtaattcaagagcataaacgatactgctaggagcagcatagttagtttcaaagtatgctttccgagcactttagcaatctccctttagaatcaggaagtgcataggctaattactatcagtcccgatatatttgttaaaggaacacctacaagatccttactggtgaccttctgtgagacactagtttgaggcactacatgtgtacttgaaaataataaagttgcatttctttatgaat

SEQ ID NO: 11 - protein, 617 aa, Mus musculus
  Protein vacuolar adenosine triphosphatase subunit A
MDFSKLPKIRDEDKESTFGYVHGVSGPVVTACDMAGAAMYELVRVGHSELVGEIIRLEGDMATIQVYEETSGVSVGDPVLRTGKPRSVELGPGIMGAIFDGIQRPLSDISSQTQSIYIPRGVNVSALSRDIKWEFIPSKNLRVGSHITGGDIYGIVNENSLIKHKIMLPPRNRGSVTYIAPPGNYDASNVVLELEFEGVKEKFSMVQVWPVRQVRPVTEKLPANHPLLTGQRVLDALFPCVQGGTTAIPGAFGCGKTVISQSLSKYSNSDVIIYVGCGERGNEMSEVLRDFPELTMEVDGKAESIMKRTALVANTSNMPVAAREASIYTGITLSEYFRDMGYHVSMMADSTSRWAEALREISGRLAEMPADSGYPAYLGARLASFYERAGRVKCLGNPEREGSVSIVGAVSPPGGDFSDPVTSATLGIVQVFWGLDKKLAQRKHFPSVNWLISYSKYMRALDEYYDKHFTEFVPLRTKAKEILQEEGDLAEIVQLVGKASLAETDKITLEVAKLIKDDFLQQNGYTPYDRFCPFYKTVGMLSNMISFYDMARRAVETTAQSDNKITWSIIREHMGEILYKLSSMKFKDPVKDGEAKIKADYAQLLEDMQNAFRSLED

SEQ ID NO: 12 - DNA, 3007 bp, Mus musculus
  cDNA vacuolar adenosine triphosphatase subunit A
gcaggtaaatttaacacaatggatttctccaagctacccaaaatccgagatgaggataaagaaagtacatttggttatgtgcatggagtctcagggcctgtggttacagcctgtgacatggcgggcgctgccatgtacgagctggtgagagtggggcacagcgagctggttggagaaattattcgattggaaggtgacatggccaccattcaggtgtatgaagaaacttctggtgtgtctgttggagaccccgtactccgcactggtaaacctcgctcggtcgagctgggtcccgggattatgggagccatttttgatggtatacagagacctctgtcggatatcagcagtcagacccaaagtatctacatccccagaggagtcaatgtgtctgctctcagcagagatatcaaatgggagtttatacccagcaaaaacctacgggttggtagtcatatcactggtggagacatttatgggattgtcaatgagaactccctcatcaaacacaaaatcatgttgcccccacgtaacagaggaagcgtgacttacatcgcgccgcctgggaattatgatgcatcgaatgtcgtcctggagcttgagtttgaaggtgtgaaggagaagttcagcatggtccaagtgtggcctgtgcggcaggtgcggcctgtcactgagaagctgcccgccaatcaccccttacttactggccagagagtcctcgatgcccttttcccgtgtgttcagggaggaactactgctatcccaggcgcctttggctgtggaaagactgtgatttcccagtctctatccaagtactccaacagtgacgtcatcatctatgtcggctgcggtgagagaggcaacgagatgtcagaagttctccgagacttccctgagctcaccatggaggttgatgggaaagcagagtccatcatgaagaggacagcgctggtagccaacacctccaacatgcctgtggctgcgagagaggcctccatctacactggaattacactatcagaatatttccgtgacatgggctaccacgtcagtatgatggccgactctacctctagatgggctgaggccctcagagaaatctctggtcgtttagctgagatgcctgcagatagtggataccctgcataccttggtgcccggctggcttctttctatgagcgagcaggcagagtgaaatgtctcggaaaccctgagagagaagggagtgtcagcattgtaggagcagtttctccacctggtggtgatttttctgatccagtcacttctgcaacgctgggtattgttcaggtgttctggggcttggataagaagttagctcagcgcaagcacttcccctctgtcaactggctcatcagctacagcaagtacatgcgtgccctggacgagtactatgacaagcacttcacggagtttgttcctctgaggaccaaagctaaggagatcctgcaggaagaaggggatctggcagaaatcgtgcagctcgtgggcaaggcctctttagcagagacggataaaatcactctggaggtagcaaaacttattaaagatgacttcctacagcaaaatgggtacactccttatgacaggttctgtccattctacaagacagtggggatgctgtccaacatgatttcattctatgacatggcccgccgagctgtggagaccactgcccagagtgacaataagatcacatggtccattatccgggagcacatgggggagattctctataaactctcctccatgaaattcaaggatccagtgaaagatggcgaggcaaagatcaaggccgactatgcacagcttcttgaagatatgcagaatgcattccgtagccttgaagactagaactgtgatttcttccctcttccacggcaagctcatacgtgtatattttcctgaatttctcatctccaacccttagcttccatattgtgcagctttgagactagtgcctatgtgtgttcttgttcattttgctgtttctttggtaggtcttataaaacaaacattcctttgttccagtgtttgaaggagctagctcccttacctttatctgaagtggtgaatgtagtgcatatgatatacattgtaagatacacattgtaacatgatccatactgtaaacttgtatgtaaggtgactaccccttccctcatctccagtaaactgtaaacaggactactgcatgtactctgttgggaatggaaggccagaactccataccgtggatggatgggtacttaggaaacaactcagcatttgtagtcagaccacttgtaacttagtggtttgttgagtaaccattttgcaggaaatacttccatttaaaaacataaaagattaatgttccaattatttgtatcaatcaggaccatttttgtggggcacttgggaactatttgtttttcaaacagacatttgcaagactgaacataatagataaatcagttacctctgaaaatgtggaaagaaagggaaaaaaagaaccaggtggtcaaacttaaattgacatcatcttgttaaagcatattttatttcactaagagaaatttaatatcagagacttttatatactcaattactaggaaacctttttttaagtacaatttaaaaatcattgaaaatgtgatccacatcatagccattcttttcctcagacttagtcagacaagcttctcagagtggtgggatggggattagaataccacagacactctgcagtgcctgcaggcagtcggccccaggacaaccactgctgtaggagtttggggccagggtggcatggttttcacaaggtacatgtgtcacgtgtttgtttgcctgttgacattctgaaaacagcaagtttaccaattgcagaaaatactttctgttttctctttcacatgctcagaaagcttctcaaaggtatctggtcacagcagcttccttcctgttatagaggtaaaaggtgttcttatatttaactggtaacaaaaga

SEQ ID NO: 13 - protein, 823 aa, Rattus norvegicus
  Protein hypoxia-inducible factor-1 alpha
MEGAGGENEKKNRMSSERRKEKSRDAARSRRSKESEVFYELAHQLPLPHNVSSHLDKASVMRLTISYLRVRKLLGAGDLDIEDEMKAQMNCFYLKALDGFVMVLTDDGDMIYISDNVNKYMGLTQFELTGHSVFDFTHPCDHEEMREMLTHRNGPVRKGKEQNTQRSFFLRMKCTLTSRGRTMNIKSATWKVLHCTGHIHVYDTSSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPASETPKPLRSSADPALNQEVALKLESSPESLGLSFTMPQIQDQPASPSDGSTRQSSPEPNSPSEYCFDVDSDMVNVFKLELVEKLFAEDTEAKNPFSAQDTDLDLEMLAPYIPMDDDFQLRSFDQLSPLESNSPSPPSVSTVTGFQQTQLQKPTITVTATATATTDESKAVTKDNIEDIKILIASPPSTQVPQEMTTAKASAYSGTHSRTASPDRAGKRVIEKTDKAHPRSLNLSVTLNQRNTVPEEELNPKTIALQNAQRKRKMEHDGSLFQAAGIGTLLQQPGDRAPTMSLSWKRVKGYISSEQDGMEQKTIFLIPSDLACRLLGQSMDESGLPQLTSYDCEVNAPIQGSRNLLQGEELLRALDQVN

SEQ ID NO: 14 - DNA, 3718 bp, Rattus norvegicus
  cDNA hypoxia-inducible factor-1 alpha
gacaccgcgggcaccgattcgccatggagggcgccggcggcgagaacgagaagaaaaataggatgagttccgaacgtcgaaaagaaaagtctagggatgcagcacgatctcggcgaagcaaagagtctgaagttttttatgagcttgctcatcagttgccacttccccacaacgtgagctcccatcttgataaagcttctgttatgaggctcaccatcagttacttacgtgtgaggaaacttctaggtgctggtgatcttgacattgaagatgaaatgaaagcacagatgaactgcttttatctgaaagccctggatggctttgttatggtgctaacagatgatggtgacatgatttacatttctgataacgtgaacaaatacatggggttgactcagtttgaactaactggacacagtgtgtttgattttacccatccatgtgaccatgaggaaatgagagaaatgcttacacacagaaatggcccagtgagaaaggggaaagaacaaaacacgcagcgaagcttttttctcagaatgaaatgtaccctaacaagccgggggaggacgatgaacatcaagtcagcaacgtggaaggtgctgcactgcacaggccacattcatgtgtatgataccagcagtaaccagccgcagtgtggctacaagaaaccgcctatgacgtgcttggtgctgatttgtgaacccattcctcatccatcaaacattgaaattcctttagacagcaagacatttctcagtcgacacagcctcgatatgaaattttcttactgtgatgaaaggattactgagttgatgggttatgagccagaagaacttttgggccgttcaatttatgaatattatcatgctttggactctgatcatctgaccaaaactcatcatgacatgtttactaaaggacaagtcaccacaggacagtacaggatgcttgcaaaaagaggtggatatgtctgggttgagactcaagcaactgttatatataatacgaagaactctcagccacagtgcattgtgtgtgtgaattatgttgtaagtggtattattcagcacgacttgattttctcccttcaacaaacagaatctgtcctcaaaccagttgaatcttcagatatgaaaatgacccagctgttcactaaagtggaatctgaggacacgagctgcctcttcgacaagcttaagaaagagcccgatgccctgactctgctagctccagcggctggggacacgatcatatcactggacttcggcagcgatgacacggaaactgaagaccaacaacttgaagatgtcccgttgtacaatgatgtaatgttcccctcttctaatgagaaattaaatataaatctggcaatgtctccattacctgcctctgaaactccaaagccacttcgaagtagtgctgatcctgcactgaatcaagaggttgcattgaagttagagtcaagcccagagtcactgggactttcttttaccatgccccagattcaagatcagccagcaagtccttctgatggaagcactagacaaagctcacctgagcctaacagtcccagtgagtactgctttgatgtggacagcgatatggtcaatgtattcaagttggaactggtggaaaaactgtttgctgaagacacagaagcgaagaatccattttcagctcaggacactgatttagacttggaaatgctggctccctatatcccaatggatgatgatttccagttacgttcctttgatcagttgtcaccattagagagcaattctccaagccctccgagtgtgagcacagttacaggattccagcagacccagttacagaaacctaccatcactgtcactgccaccgcaactgccaccactgatgaatcaaaagcagtgacgaaggacaatatagaagacattaaaatactgattgcatctccaccttctacccaagtacctcaagaaatgaccactgctaaggcatcagcatacagtggtactcacagtcggacagcctcaccagacagagcaggaaagagagtcatagaaaaaacagacaaagctcatccaaggagccttaacctatctgtcactttgaatcaaagaaatactgttcctgaagaagaattaaacccaaagacaatagctttgcagaatgctcagaggaagcgaaaaatggaacatgatggctccctttttcaagcagcaggaattggaacgttactgcagcaaccaggtgaccgtgcccctactatgtcgctttcttggaaacgagtgaaaggatacatatctagtgaacaggatggaatggagcagaagacaatttttttaataccctctgatttagcatgtagactgctggggcagtcaatggatgagagtggattaccacagctgaccagttacgattgtgaagttaatgctcccatacaaggcagcagaaacctactgcagggtgaagaattactcagagctttggatcaagttaactgagcttttcctaatctcattcctttgattgttaatttttgtgttcagttgttgttgttgtctgtggggtttcgtttctgttggttgttttggacactggtggctcagcagtctatttatattttctatatctcatttagaggcctggctacagtactgcaccaactcagatagtttagtttgggccccttcctccttcattttcactgatgctctttttaccatgtccttcgaatgccagatcacagcacattcacagctccccagcatttcaccaatgcattgctgtagtgtcgtttaaaatgcacctttttatttatttatttttggtgagggagtttgtcccttattgaattatttttaatgaaatgccaatataattttttaagaaggcagtaaatcttcatcatgatgataggcagttgaaaattttttactcatttttttcatgttttacatgaaaataatgctttgccagcagtacatggtagccacaattgcacaatatattttcttaaaaataccagcagttactcatgcatatattctgcatttataaaactagtttttaagaagaaactttttttggcctatggaattgttaagcctggatcatgatgctgttgatcttataatgattcttaaactgtatggtttctttatatgggtaaagccatttacatgatatagagagatatgcttatatctggaaggtatatggcatttatttggataaaattctcaattgagaagttatctggtgtttctttactttaccggctcaaaagaaaacagtccctatgtagttgtggaagcttatgctaatattgtgtaattgatattaaacattaaatgttctgcctatcctgttggtataaagacattttgagcatactgtaaacaaaaaaatcatgcattgttagtaaaattgcctagtatgttaatttgttgaaaatacgatgtttggttttatgcactttgtcgctattaacatcctttttttcatatagatttcaataattgagtaattttagaagcattattttagaaatatagagttgtcatagtaaacatcttgtttttttttctttttttctatgtacattgtataaatttttcattcccttgctctttgtagttgggtctaacactaactgtactgttttgttatatcaaataaacatcttctgtggaccaggaaaaaaaaaaaaaaaaaaaaaaaaaaa

SEQ ID NO: 15 - protein, 227 aa, Rattus norvegicus
  Protein cytochrome c oxidase subunit II
MAYPFQLGLQDATSPIMEELTNFHDHTLMIVFLISSLVLYIISLMLTTKLTHTSTMDAHEVETIWTILPAVILILIALPSLRILYMMDEINNPVLTVKTMGHQWYWSYEYTDYEDLCFDSYMIPTNDLKLGELRLLEVDNRVVLPMELPIRMLISSEDVLHSWPIPSLGLKTDAIPGRPNQATVTSNRLGLFYGQCSEICGSNHSFMLIVLEMVPLKYFENWSASMI

SEQ ID NO: 16 - DNA, 686 bp, Rattus norvegicus
  cDNA cytochrome c oxidase subunit II
acatggcttacccatttcaacttggcttacaagacgccacatcaccaatcatagaagaacttacaaactttcatgaccacaccctaataattgtattcctcatcagctccctagtactttatattatttcactaatactaacaacaaaactaacacacacaagcacaatagacgcccatgaagtagaaacaatttgaacaattctcccagctgtcattcttattctaatcgcccttccctccctacgaattctatacataatagacgagattaataacccagttctaacagtaaaaactataggacaccaatgatactgaagctatgaatatactgactatgaagacctatgctttgactcctacataatcccaaccaatgacctaaaactaggtgaacttcgcttattagaagttgataatcgggtagtcttaccaatagaacttccaattcgtatactaatctcatccgaagacgtcctgcactcatgacccatcccttcactagggttaaaaaccgacgcaatccccggccgcccgaaccaagctacagtcacatcaaaccgactaggtctattctatggccaatgctctgaaatttgcggctcaaatcacagcttcatactcattgtactagaaatagtgcctctaaaatatttcgaaaactgatcagcttctataatttaa

SEQ ID NO: 17 - protein, 260 aa, Rattus norvegicus
  Protein HYDROXYACYLGLUTATHIONE HYDROLASE
MKIELLPALTDNYMYLIIDEDTQEAAVVDPVQPQKVIETVKKHRVKLTTVLTTHHHWDHAGGNEKLVKLEPGLKVYGGDDRIGALTHKVTHLSTLEVGSLSVKCLSTPCHTSGHICYFVSKPGSSEPSAVFTGDTLFVAGCGKFYEGTADEMYKALLEVLGRLPPDTKVICGHEYTVNNLKFARHVEPGNTAVQEKLAWAKEKNAIGEPTVPSTLAEEFTYNPFMRVKEKTVQQHAGETDPVTTMRAIRREKDQFKVPRD

SEQ ID NO: 18 - protein, 504 aa, Homo sapiens
  Protein 26S PROTEASOME SUBUNIT S5B
MAAQALALLREVARLEAPLEELRALHSVLQAVPLNELRQQAAELRLGPLFSLLNENHREKTTLCVSILERLLQAMEPVHVARNLRVDLQRGLIHPDDSVKILTLSQIGRIVENSDAVTEILNNAELLKQIVYCIGGENLSVAKAAIKSLSRISLTQAGLEALFESNLLDDLKSVMKTNDIVRYRVYELIIEISSVSPESLNYCTTSGLVTQLLRELTGEDVLVRATCIEMVTSLAYTHHGRQYLAQEGVIDQISNIIVGADSDPFSSFYLPGFVKFFGNLAVMDSPQQICERYPIFVEKVFEMIESQDPTMIGVAVDTVGILGSNVEGKQVLQKTGTRFERLLMRIGHQSKNAPVELKIRCLDAISSLLYLPPEQQTDDLLRMTESWFSSLSRDPLELFRGISSQPFPELHCAALKVFTAIANQPWAQKLMFNSPGFVEYVVDRSVEHDKASKDAKYELVKALANSKTIAEIFGNPNYLRLRTYLSEGPYYVKPVSTTAVEGAE

SEQ ID NO: 19 - protein, 1128 aa, Homo sapiens
  Protein Nck-associated protein 1 (Nap 1)
MSRSVLQPSQQKLAEKLTILNDRGVGMLTRLYNIKKACGDPKAKPSYLIDKNLESAVKFIVRKFPAVETRNNNQQLAQLQKEKSEILKNLALYYFTFVDVMEFKDHVCELLNTIDVCQVFFDITVNFDLTKNYLDLIITYTTLMILLSRIEERKAIIGLYNYAHEMTHGASDREYPRLGQMIVDYENPLKKMMEEFVPHSKSLSDALISLQMVYPRRNLSADQWRNAQLLSLISAPSTMLNPAQSDTMPCEYLSLDAMEKWIIFGFILCHGILNTDATALNLWKLALQSSSCLSLFRDEVFHIHKAAEDLFVNIRGYNKRINDIRECKEAAVSHAGSMHRERRKFLRSALKELATVLSDQPGLLGPKALFVFMALSFARDEIIWLLRHADNMPKKSADDFIDKHIAELIFYMEELRAHVRKYGPVMQRYYVQYLSGFDAVVLNELVQNLSVCPEDESIIMSSFVNTMTSLSVKQVEDGEVFDFRGMRLDWFRLQAYTSVSKASLGLADHRELGKMMNTIIFHTKMVDSLVEMLVETSDLSIFCFYSRAFEKMFQQCLELPSQSRYSIAFPLLCTHFMSCTHELCPEERHHIGDRSLSLCNMFLDEMAKQARNLITDICTEQCTLSDQLLPKHCAKTISQAVNKKSKKQTGKKGEPEREKPGVESMRKNRLVVTNLDKLHTALSELCFSINYVPNMVVWEHTFTPREYLTSHLEIRFTKSIVGMTMYNQATQEIAKPSELLTSVRAYMTVLQSIENYVQIDITRVFNNVLLQQTQHLDSHGEPTITSLYTNWYLETLLRQVSNGHIAYFPAMKAFVNLPTENELTFNAEEYSDISEMRSLSELLGPYGMKFLSESLMWHISSQVAELKKLVVENVDVLTQMRTSFDKPDQMAALFKRLSSVDSVLKRMTIIGVILSFRSLAQEALRDVLSYHIPFLVSSIEDFKDHIPRETDMKVAMNVYELSSAAGLPCEIDPALVVALSSQKSENISPEEEYKIACLLMVFVAVSLPTLASNVMSQYSPAIEGHCNNIHCLAKAINQIAAALFTIHKGSIEDRLKEFLALASSSLLKIGQETDKTTTRNRESVYLLLDMIVQESPFLTMDLLESCFPYVLLRNAYHAVYKQSVTSSA

SEQ ID NO: 20 - DNA, 4218 bp, Homo sapiens
  cDNA Nck-associated protein 1 (Nap 1)
cggcggcaccagcaccaccatgtcgcgctcagtgctgcagcccagtcagcagaagctggcggagaagctcaccatcctcaacgaccggggcgtcggcatgctcacccgcctctacaacatcaagaaggcatgtggagaccccaaggcaaaaccatcctatcttatcgacaaaaacctggaatctgctgtgaaattcatagtcagaaaattccctgctgtagaaacccgcaacaacaatcaacagcttgcacaactacagaaagaaaaatcagagattctgaaaaatctggcattatattacttcacatttgtagatgttatggaatttaaggaccatgtttgtgaattgctgaatactattgacgtttgccaagtcttctttgatattactgtaaactttgatttaacaaagaactacttagatttaattataacctatacaacactaatgatactgctgtctcgaattgaagaaaggaaggcaatcattggattatacaactatgcccatgaaatgactcatggagcaagtgacagagaatacccacgccttggccagatgattgtggattatgaaaaccctttaaagaagatgatggaagaatttgtaccccatagcaagtctctttcagatgcactaatttctcttcaaatggtatatcctcgaaggaatctttcagctgaccagtggagaaatgcccagttattgagcctcatcagtgcacctagtacaatgcttaatccagcacagtccgacactatgccttgtgaatacctctctttggatgcaatggaaaagtggattatctttggctttattttgtgccatgggatcctaaatactgacgctacagcactgaacctttggaaactagctcttcaaagtagctcttgcctctctctctttcgggatgaagttttccacattcacaaagctgcagaagacttatttgtaaacatacgaggctataataaacgtattaatgacataagagaatgcaaggaggcagccgtgtcacatgctggttcaatgcacagagaaagacgcaagtttttaagatctgcactgaaggaattggctactgtcctctctgatcaacctggattgctaggtcccaaggcactttttgtttttatggcattatcctttgcccgtgatgaaatcatctggctacttcgtcatgcagataacatgccaaagaagagtgcagacgactttatagataagcacattgctgaattaatattttacatggaagaacttagagcacatgtgaggaaatacggacctgtaatgcagaggtattacgtgcagtacctttctggctttgatgctgttgtcctcaatgaactcgtgcagaatctttctgtttgccctgaagatgaatcaatcatcatgtcctcttttgttaacactatgacttccctaagtgtaaaacaagttgaagatggggaagtatttgatttcagaggaatgagattagattggtttaggttacaggcatatactagtgtctcaaaggcttcacttggccttgcagatcacagagaacttgggaagatgatgaatacaataatttttcatacaaaaatggtagattccttggtggaaatgttggtggaaacatcagatctctccatattttgtttttatagtcgtgcttttgagaagatgtttcaacagtgtttggagttaccctctcaatcaagatactcaattgcatttccactactttgcactcattttatgagttgcacgcatgaactatgtccagaagagcgacatcatattggagatcgcagtctttccttatgtaatatgttcctagatgaaatggccaaacaagctcgaaatctcatcactgatatttgcacagaacagtgtacccttagtgaccagttgctacccaagcattgtgccaaaactatcagtcaagcagtgaataagaaatcaaaaaagcagactggtaagaaaggggaacctgaaagggagaaaccaggtgttgagagcatgaggaaaaacaggctggttgtgaccaaccttgataaattgcacactgcactttctgagttatgcttctctataaattatgtaccaaacatggtggtatgggaacatacctttaccccacgagaatatttgacttctcatctggaaatacgctttaccaagtcaattgttgggatgactatgtataatcaagccacacaggaaattgcaaaaccttcagaacttctaacaagtgtaagagcatacatgaccgtactccagtcaatagaaaactatgtgcagattgatattacaagagtatttaataatgtgcttcttcaacaaacacaacatttagacagtcatggagagccaaccattacaagtctatacacaaattggtatttggaaactttgttacgacaagtcagcaatggccatatagcatattttcctgcaatgaaagcgtttgtgaacttacctacagaaaatgaattaacattcaatgcagaggaatattctgacatatcagaaatgaggtcattatcagaactactaggcccatatggtatgaagtttctaagtgaaagccttatgtggcatatttcatcacaagttgctgaacttaagaaacttgtggtggagaatgttgatgtgttaacacaaatgaggaccagctttgacaaaccagaccagatggctgcactgtttaaaagattatcatctgttgacagtgtcttgaagaggatgacaataattggtgtaattttatccttccgatcattggcacaagaagcacttagagatgtgttatcctaccacattccttttcttgtaagttcaattgaagattttaaggatcacattccaagggaaactgatatgaaggttgcaatgaatgtgtatgagttatcatcagctgccggattaccttgtgagattgatcctgcattggtcgtagctctttcttcacaaaaatcggaaaacattagtccagaagaagagtataaaattgcctgccttctcatggtgtttgtggcagtttctttgccaacactggccagtaatgtgatgtctcagtacagccctgctatagaagggcattgcaacaacatacattgcttggccaaagccatcaaccagattgctgcagctttgtttacaattcacaaaggaagcattgaagaccgtcttaaagaatttctggcgcttgcatcctccagtctactgaaaattggccaggagacagataaaactacaacaagaaatagagaatctgtttatttactgctagatatgattgtacaagaatctccattccttacaatggatcttttggaatcttgttttccttatgtcttgctgagaaatgcataccatgctgtctacaaacaaagtgttacatcttctgcataaaattacctacttaatcaagataagcacgcatttttgttgccttggttttacctgtagactgtggaactattttaccttaagacctgaaaaagttttgtggattataaatttctttcatacggttgtattttctgatcattggtttcttaatatggttgtactacagtatacttggttgatttaggttgcacattcactgaattcactgagattattcctataattttaaagtatcatttatttgaaaaacatacattatcaacatgtttttgatatttgataatgaaaaaaatctttgcttgtttatttctgaaaaagaactgtatttagtgattattttagatagtgatattatagcattcatctgtgtgtaaattatttcatatagggaagagttctgatctgtacctatggttcttattgaaaacaacattggatgtgcatttctgtgatgttatgaatacatttctactttattttgaaacatttgccaaactaaatactgtaacactgtataacatttaaaaatgttaaagaactgcttagtattagaagcagatcatttcccaaaattctaagagcagcagcatatgttgttgcttgtataaagcctagcgataatttttagactaacttccatggtgccctgttggcattagcactaccattgtaccctgctgtataataaacaatcttagacatttatcaactgttgatacaaatgttagtccctaaccactttttatatatgttttaaatttttgaaattcaagtgtaccttccataacataaaataaacactagactgtatc

SEQ ID NO: 21 - protein, 166 aa, Mus musculus
  Protein Cofilin
MASGVTVNDEVIKVFNDMKVRKSSTQEEIKKRKKAVLFCLSDDKRQIIVEEAKQILVGDIGDTVEDPYTSFVKLLPLNDCRYALYDATYETKESKKEDLVFIFWAPESAPLKSKMIYASSKDAIKKKFTGIKHEWQVNGLDDIKDRSTLGEKLGGSVVVSLEGKPL

SEQ ID NO: 22 - DNA, 2974 bp, Mus musculus
  cDNA Cofilin
cgcccggctgcagctcccggcgtgccctgcactctctgctgcccgccgccgacccctccttcttctcgtcccagtgccaccgagccggagtccgagccaccgccgccgcagccacttcagccgcgggcactatggcatctggagttacagtgaatgatgaagtcatcaaagtttttaatgatatgaaagtaagaaaatcttctacacaggaggagatcaaaaaaagaaagaaagcagttctcttctgtttgagcgatgacaaaagacaaataattgtagaggaagccaagcagatcttggtgggtgacattggtgacactgtagaggacccctacacatcttttgtgaagttgttgcctctgaatgattgccgatatgctttgtacgatgccacgtacgaaacaaaagagtctaagaaagaagacctagtatttatattctgggctcctgaaagtgcaccgttaaaaagcaagatgatttatgctagctctaaagatgccattaaaaagaaatttacaggtattaaacatgagtggcaagtaaatggcttggacgatattaaggaccgctcgacgctgggagagaaactggggggcagtgttgtagtttcccttgaaggaaagccactataaaataatagccaagtgccatttgatcttaaggggcttacacgtatctctccagctcagtccactggaattgtattaggttttgttttttttgtttattcccttttcactggtcccgttcgtgaatgagtgaatataagaagcctgtcagtattgccatgagactgtttcatatggttacttttctgtattcccaaggaatgccttcctgtcttattttagccaaaacaaactggttccatgccttccttgcagtgagcgttacaatggatgtggttgtcaatgtgaatagcttagagtactacaaagggtaagctaactgaatgccttgaaaatattatccactggtcggtcatatgggagacttgtttcagtattatttatagttgcacttgattaccgttctctgaggcactggagccttcatacacctcacctgccttggcaagcctatttttgtgacctggcagcacagatttaacactattcgttaaaagcacttttttttaatgcgtttaatcccttataaagaatgccaattaagttttattacctgtcatcaatttatcctagtatctcagtgttcattcttcttgccttcatatttttttcaaagaaacagctgtgctaatgtctttggtttcccgatgagtgtacactactgtataatttatgtttaccatatgagtcttgaaacactacagatattttgaatatcagtcatgatggcaatttctgtataaaagagccttaaatggaacattgttttgagatcaaactccctaccctcacaaaagtggccacgttgcaataaaaattgtggcagattacagaatgttgccttgttttccttggaaattttgcaaattgttatgtgaaattttagggtaacggtgattaagctctgcactggtatttggaattttttttcctttaatctttggtttaaaaacatcttaaaatcacttatatacaatcattaaaagagtggtaattttataaatgcttatttatgttataaaatggagatcagaaaaaaattctttttgcactttggcctatccagtatcttatttattctctagataagctaggatattaatccagagttacattactgagaattgagtagtataagtaggatgtttttattacttggtcataatgaaaataatttgtaaaatgtcattcgaaggttaatgatgattgtgatgtttaggaatgtttgtctcagccacagttccttcatagcttttccaaaatgaattgggaaaaaaaatcgtatagcagtcttaaagcttagtaatggaacttggctgtggcccagagctttctccttatagagaatttgatctgctccgtgtgtgctctctgctattagccggagctatttatggcaaacacatgcttttgtatcttgtcatagtcatccacagatggcaaaactggacttgattctactggcatgtaagacaggcgtgctagtgagcagtcgtgtgtggctctggactctgaccccagagctctgaagaatgctcttatcagaagataggaaatgaaaatatccttttttaaaatatgtggaagtaatttgggtataattagtttttttctaccttttggaaagttgtttttttgttgtttttttttttttcccagatgagaacattaacatagtggttaaatgtctaggcttccatttaaaactacacaaatgacttgggatctttttagcactaaggaatttgatttcagccttccagctgttgctgtgagttgttccagacctttctgtggctttttggtaaggctgcttagaagcatgagaagcatgagaatggtaatgtgtgctaaacctatgtttaaccaatctttgcaccaaaggactttttcaccaatttattttgttattcttctaaatattaagtgatttctaaaaggtaaagggtgaccttttgtttttatatctaatttctcaatttctttatatgcatttttagaataatttgagagattaaatgctgcttgaaactattatactttgagttttagattggccaaatacattaatgtagttaaattcatctttaaagtacacatatgtgcctagagccaaaaaataataatgatttaatttatgaccttatgttgagactaatttcacatcttattttacagtcatttacagtgaaacaatgttccagctagctttaaaagctatacggtgctaattagtaaaatattgagggcaatattttactgctagcttgcaaaattataagtgttttaaaaataaaatacatgaaaaaaaaaaaaaaaaaaaaaaaaaaaaaaaaaaaaaaaaa

SEQ ID NO: 23 - protein, 117 aa, Homo sapiens
  Protein Ganglioside expression factor 2
MKWMFKEDHSLEHRCVESAKIRAKYPDRVPVIVEKVSGSQIVDIDKRKYLVPSDITVAQFMWIIRKRIQLPSEKAIFLFVDKTVPQSSLTMGQLYEKEKDEDGFLYVAYSGENTFGF

SEQ ID NO: 24 - DNA, 975 bp, Rattus norvegicus
  cDNA Ganglioside expression factor 2
cccgcctgccgagtagtcgtcgctgccgccgccgcctccgttgttgttgtggtcgcttcgccgaagtctgcggctcaaagagccggctccgtcgcttcccgccgccatgaagtggatgtttaaggaggaccactcgctggaacacagatgcgtggaatccgcgaagatcagagcgaaataccccgaccgggttccggtgatcgttgagaaagtctctggctctcagattgttgacattgacaagaggaagtacttggtcccatctgacatcactgtggctcagttcatgtggatcatcaggaaaaggatccagcttccttctgagaaggccatcttcttgtttgtggacaagacagtcccacagtccagcctaactatgggacagctttacgagaaggaaaaagatgaagatggattcttgtatgtggcctacagcggagagaacacttttggcttctgagcccttgctgggctaggtgcacccttcctgcttgtgtatcctgtaaataactggctgttctcagttactccgccggagcctccacacagacctactagtgcatttgtaactggatttatttcttaatatattggaaggttttgttttccttagattagtaaattatcatacagagttttattttcagttttcttttgtgcactgtcctcatggctatatgctccaaggaacctgtcctccggaatcacatttaatgaagatacttccgaaatgaagggcggtaggtgtggtattaaagtgacaaggagggatgacgcattgttctggattatgttcggagtgttagacggctaagtattaaaagcccccaaattaaatccttagcaatcagaacacttgcttcactagattttgccaactgcaaatcatgttggactgagctaatctgttctttctgagactataaggtaaatgattaacaataaagcctccatgtaaaaggcaaaaaaaaaaaa

SEQ ID NO: 25 - protein, 1377 aa, Homo sapiens
  Protein HMG1/2
IPMVVSDFDLPDQQIEILQSSDSGCSQSSAGDNLSYEVDPETVNAQEDSQMPKESSPDDDVQQVVFDLICKVVSGLEVESASVTSQLEIEAMPPKCSDIDPDEETIKIEDDSIQQSQNALLSNESSQFLSVSAEGGHECVANGISRNSSSPCISGTTHTLHDSSVASIETKSRQRSHSSIQFSFKEKLSEKVSEKETIVKESGKQPGAKPKVKLARKKDDDKKKSSNEKLKQTSVFFSDGLDLENWYSCGEGDISEIESDMGSPGSRKSPNFNIHPLYQHVLLYLQLYDSSRTLYAFSAIKAILKTNPIAFVNAISTTSVNNAYTPQLSLLQNLLARHRISVMGKDFYSHIPVDSNHNFRSSMYIEILISLCLYYMRSHYPTHVKVTAQDLIGNRNMQMMSIEILTLLFTELAKVIESSAKGFPSFISDMLSKCKVQKVILHCLLSSIFSAQKWHSEKMAGKNLVAVEEGFSEDSLINFSEDEFDNGSTLQSQLLKVLQRLIVLEHRVMTIPEENETGFDFVVSDLEHISPHQPMTSLQYLHAQPITCQGMFLCAVIRALHQHCACKMHPQWIGLITSTLPYMGKVLQRVVVSVTLQLCRNLDNLIQQYKYETGLSDSRPLWMASIIPPDMILTLLEGITAIIHYCLLDPTTQYHQLLVSVDQKHLFEARSGILSILHMIMSSVTLLWSILHQADSSEKMTIAASASLTTINLGATKNLRQQILELLGPISMNHGVHFMAAIAFVWNERRQNKTTTRTKVIPAASEEQLLLVELVRSISVMRAETVIQTVKEVLKQPPAIAKDKKHLSLEVCMLQFFYAYIQRIPVPNLVDSWASLLILLKDSIQLSLPAPGQFLILGVLNEFIMKNPSLENKKDQRDLQDVTHKIVDAIGAIAGSSLEQTTWLRRNLEVKPSPKIMVDGTNLESDVEDMLSPAMETANITPSVYSVHALTLLSEVLAHLLDMVFYSDEKERVIPLLVNIMHYVVPYLRNHSAHNAPSYRACVQLLSSLSGYQYTRRAWKKEAFDLFMDPSFFQMDASCVNHWRAIMDNLMTHDKTTFRDLMTRVAVAQSSSLNLFANRDVELEQRAMLLKRLAFAIFSSEIDQYQKYLPDIQERLVESLRLPQVPTLHSQVFLFFRVLLLRMSPQHLTSLWPTMITELVQVFLLMEQELTADEDISRTSGPSVAGLETTYTGGNGFSTSYNSQRWLNLYLSACKFLDLALALPSENLPQFQMYRWAFIPEASDDSGLEVRRQGIHQREFKPYVVRLAKLLRKRAKKNPEEDNSGRTLGWEPGHLLLTICTVRSMEQLLPFFNVLSQVFNSKVTSRCGGHSGSPILYSNAFPNKDMKLENHKPCSSKARQKIEEMVEKDFLEGMIKT

SEQ ID NO: 26 - DNA, 1000 bp, Homo sapiens
  cDNA HMG1/2
tttttttttttttttttttgaaaaaaaaaatgggtagtgtatattttgcaggtttaagacaactcaggacaataaaaacaatggactttacatgtgtatatatatagctctcttaggcaccataatcagtatgagccaacaatatttaaacttgattcaggccacattcagacatttgctcttatatacaaatatttaaattaaatacaatctgaaatgtgttctgttacatacaaaaaaggaaaaactatacaacgcagagcagtgtgtgtgttttaaataattacatttacatgtaagctaaatggaaccagcaatggtgctcaagtttttatcatcccttccagaaaatctttttctaccatctcttctattttttgcctggctttgctggaacatggtttgtggttctccagtttcatgtccttattagggaaggcatttgagtagaggataggactccctgagtgtcctccacatcggcttgtgactttgctgttgaagacttgactgagcacattgaagaacggcaggagctgctccatactgcgcacggtgcagatggtgagcagcaagtgccctggctcccaacccaatgttctccctgagttgtcttcctctggatttttctttgctcttttccgaagaagttttgctagtcgtaccacgtaaggtttaaattctcgttgatgtataccctgccttctgacttccaaacctgaatcatctgaggcttctggaataaaggcccatcggtacatctgaaactgaggaaggttttcagagggcaatgcgagagccaaatccaaaaatttgcaagcagagagatagaggtttaaccaccgctggctgttatatgaagtagagaagccattacctcctgtgtacgttgtctccagaccagccacagagggccctgaagtccgtgaaatatcttcatcagcagtgagttcctgctccatcagtaaaaatacttgtacaagttctgtaatcatggtag

SEQ ID NO: 27 - protein, 896 aa, Mus musculus
  Protein Mouse phosphoprotein (F1-20)
MSGQTLTDRIAAAQYSVTGSAVARAVCKATTHEVMGPKKKHLDYLIQATNETNVNIPQMADTLFERATNSSWVVVFKALVTTHHLMVHGNERFIQYLASRNTLFNLSNFLDKSGSHGYDMSTFIRRYSRYLNEKAFSYRQMAFDFARVKKGADGVMRTMVPEKLLKSMPILQGQIDALLEFDVHPNELTNGVINAAFMLLFKDLIKLFACYNDGVINLLEKFFEMKKGQCKDALEIYKRFLTRMTRVSEFLKVAEQVGIDKGDIPDLTQAPSSLMETLEQHLNTLEGKKPGNNEGSGAPSPLSKSSPATTVTSPNSTPAKTIDTSPPVDIFATASAAAPVSSAKPSSDLLDLQPDFSGAAAGAAAPVVPPSGGATAWGDLLGEDSLAALSSVPCEAPISDPFAPEPSPPTTTTEPASASASTTTAVTAVTTEVDLFGDAFAASPGEAPAASEGATAPATPAPVAAALDACSGNDPFAPSEGSAEAAPELDLFAMKPPETSAPVVTPTASTAPPVPATAPSPAPTAVAATAATTTAAAAATTTATTSAAAATTAAAPPALDIFGDLFDSAPEVAAAPKPDAAPSIDLFGTDAFSSPPRGASPVPESSLTADLLSVDAFAAPSPASTASPAKAESSGVIDLFGDAFGSGASETQPAPQAVSSSSASADLLAGFGGSFMAPSTTPVTPAQNNLLQPSFEAAFGTTPSTSSSSSFDPSGDLLMPTMAPSGQPAPVSMVPPSPAMAASKGLGSDLDSSLASLVGNLGISGTTSKKGDLQWNAGEKKLTGGANWQPKVTPATWSAGVPPQGTVPPTSSVPPGAGAPSVGQPGAGFGMPPSGTGMTMMSQQPVMFAQPMMRPPFGAAAVPGTQLSPSPTPATQSPKKPPAKDPLADLNIKDFL

SEQ ID NO: 28 - DNA, 4260 bp, Mus musculus
  cDNA Mouse phosphoprotein (F1-20)
gggacaggggccgggccggggctacaggcatcgcggcccggggacaccagggcggtgcgtgtctgcacccagctacctcgcgcggcgtccgggctgcggtgctcctggggcgggaagaggaggcggtagacgcggccggtgaagatgtcgggccaaacgctcacggatcggatcgccgccgctcagtacagcgtgactggctctgctgtagcaagagcagtctgcaaagccaccactcatgaggtgatgggccccaagaagaagcacctggactatttgatccaggctaccaatgagaccaatgtcaatatccctcagatggccgacaccctctttgagcgggcgacaaacagtagctgggtggtggtatttaaagctttagtgaccacacaccatctcatggtgcatggaaatgagagatttattcagtatttggcctctaggaatacgctattcaatctcagcaactttctagataaaagtggatcccacggttatgatatgtctacgttcatacggcgttacagtagatacttgaatgaaaaggctttctcctacagacagatggcatttgactttgccagagtgaagaaaggggctgacggtgtcatgaggacgatggttcctgaaaagctcctgaagagtatgccaatcctgcaggggcagatcgatgcactgctggagtttgatgtgcatccaaatgaactaaccaatggtgtcataaatgctgcatttatgcttcttttcaaagatcttatcaaactgtttgcttgctacaatgacggcgtcattaacttacttgaaaaattttttgagatgaagaaaggtcaatgcaaagacgcgctagaaatttacaagcgatttctaactagaatgacgagggtgtccgaattcctcaaggtcgccgagcaagttggtattgataaaggcgacattcccgacctcacgcaggctcccagcagtcttatggagacccttgaacaacatctaaataccctagaaggaaagaaacctggaaacaatgaaggatctggtgctccctctccactaagtaagtcttctccagccacaactgttacatctcctaattctacaccagctaaaactatcgacacatccccgccagttgacatatttgcaacagcatccgcggctgccccagtcagctctgctaagccatcaagcgatctccttgatcttcagcccgacttctctggagcggctgcgggggcagcagcacctgtagtgcctccttctgggggtgcgaccgcctggggagaccttttgggagaggattccttggctgcactttcctctgttccctgtgaagcaccgatttcagacccatttgcaccagagccttcccctcctactacaaccactgagcctgcttcagcctctgcctcgaccaccacagctgtgacggctgtcactacggaagtggatctctttggagatgcctttgcagcttctcctggggaggcccctgcagcatccgaaggggctaccgcaccagctaccccggccccagtggctgcagctcttgatgcatgctcaggaaatgacccttttgccccatctgaaggtagcgcagaggctgcacctgagctggacctctttgcaatgaagccacctgagaccagcgctcctgtagttacccctacagctagcacagcccctccagttcccgcaactgctccttctcctgctcccacggctgtggcagccactgctgccaccaccaccgccgccgctgcagctaccaccactgccaccacctctgctgctgctgccaccaccgccgctgctcctcctgctctagatatctttggtgatttgtttgattctgctcctgaagttgctgcagcacctaagccagacgcggctcctagcatagacctgtttggcacagatgctttctcctccccgccacgaggggcctctccggtgcctgagagttctctcactgctgacctcttatctgtggacgcatttgcagcgccgtctcctgcatccactgcctctcctgcaaaggcggagtcctcgggtgtcatagacctttttggggatgcgtttggaagtggtgcttctgaaacccagccagcaccacaggctgtttctagttcatcagcatcggcagatctactagctggatttgggggttctttcatggccccttctacaacgccagtgactccagctcagaataacctgctgcaacccagtttcgaggcagcttttggaacgacgccttcgacttcaagcagcagctcttttgacccatcagtgtttgatggtttaggcgatcttctgatgccaaccatggcaccatccgggcagcctgcccctgtctcaatggtcccacccagtcctgcaatggcagccagcaaaggcctcggaagtgaccttgactcgtctctggccagtttagtaggcaatcttgggatttctggtaccacatcaaaaaagggagatctccagtggaatgctggggagaaaaagctgactggtggagccaactggcaaccgaaagtcactccagccacatggtcagcgggtgttcccccgcaaggcactgttccaccaaccagctcagtccctccgggtgccggggccccgtccgttgggcaacctggagcaggatttggaatgcctccttcagggacaggcatgaccatgatgtctcagcagccagtcatgtttgcacagcccatgatgaggccaccctttggagctgcagctgtgcccggcacacagctttctccaagccctacacctgccactcagagtcccaagaaacctccagccaaggacccgttagcggatcttaacatcaaggatttcttgtaaacaatttaagctgcaatttttgtgactgaataggaaaaaactaacctgagtttggaaacttcagataagattgatgctcagtttcaaagtgagccaccagtaccaaacccagtgtgacgcgtaacttcctctcccaagcacacaggccagctgtggcagtgaacattaggaatgtgtactctttagctgttaccctgctcttccagctcgtagcgtattcgggttctttgtgtgttgtacgaagtaaaccatgagtgaatgaatggttccaatgcctttagtgcttttctggacgttgcccgtggacggaggatgacgcagctgttctgtggtgagccatttggaaagatgtgtctgtgttttcgaaaggtttcgatgtatatataacttttggacaaactcaagtcctcccatgaactttctcttctcttctgtacctctgttacaagcgtaatgtgatactatcagctagtgagacaaacactcttaactatacaaaaactttttcggtgtggagtacatttttccaatcacagaaacttccaacttgttgtgagaaatgtttatttttgtgcactgtatatgttaagaaattttattttaaaaaaaaatgaagtctaatgtccataataaaacttctccttgatgaagctaccttatcaagaatgagaaaaaccatatgagaagtccatatttatcactgctatattaagatatatattttaattatatttgcagggtttgcatctaaattgacctatttattcatttgtgatcaaaggcaccgaaaagtggagcttgtttctattgtgtctgtgacaatgcagttagagatgtgctgtttctgtgactacgaacgtgccccaagagacatctgtaacttaaagagaactgcaaatattttttatttcagtgtgggtttacgtacatctgttcagttagtatttctttgtgtgttctatagagtagtgtttctccatccttcaattgagctcaaagtgacttctattgtacctttgtgataggattgaaaccaattcagcgaattgtatcttttaatgtacataatgtattctttgattttcagtttgttgtcatactgtctgtgccgatggcttggctgaagattttgatgcgtacacaaggtcactgttgatcagtgttgtttagtggcttggcagctctttgtaaaagcatattgggttggaaaggcatttgcctatttttcaaattatttaatagatgtatggtaccctttaaagtggttgtatctgaatttactgtggggataacatacactgtaatgggggaaaattacctaaaaccaatttcaaaatggcttttctttgtattttggtttaaaaacccagtgcatgtctgccctctgagatgcaataaacaccttgaacc

SEQ ID NO: 29 - protein, 196 aa, Rattus norvegicus [sequence omitted]

SEQ ID NO: 30 - DNA, 701 bp, Rattus norvegicus
  cDNA ribosomal protein L 19
ctttcctttcgctgctgcgtctgcagccatgagtatgcttaggctacagaagaggcttgcctctagcgtcctccgctgtggtaaaaagaaggtgtggttggaccccaatgaaaccaacgaaatcgccaatgccaactctcgtcaacagatcaggaagctgatcaaagatggcctgatcatccggaagcctgtgactgtccattcccgggctcgatgccggaagaacaccttggcccgacggaagggcaggcatatgggcatagggaagaggaagggtactgccaacgctcggatgcccgagaaggtgacctggatgcgaaggatgaggatcctgcgccggcttctcaggagataccgggaatctaagaagattgaccgtcatatgtatcacagcctgtacctgaaggtcaaagggaatgtgttcaaaaacaagcggattctcatggagcacatccacaaactgaaggcagacaaggcccgcaagaagctactggctgaccaggctgaggctcgcaggtctaagaccaaggaagcacgaaagcgccgggaggagcgcctccaagccaagaaggaggagatcatcaagactctgtccaaggaggaagagaccaagaaatgaagcgtccctcgtgtctgtacatagtggctaggctatggcccacatggatcagtcattaaaataaaacaagccttcgtccttg

SEQ ID NO: 31 - protein, 245 aa, Rattus norvegicus
  Protein 14-3-3 zeta-isoform
MDKNELVQKAKLAEQAERYDDMAACMKSVTEQGAELSNEERNLLSVAYKNVVGARRSSWRVVSSIEQKTEGAEKKQQMAREYREKIEMELRDICNDVLSLLEKFLIPNASQPESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 32 - DNA, 1687 bp, Rattus norvegicus
  cDNA 14-3-3 zeta-isoform
cccagagactgccgagccgggtccgtgtgccgccacccactccggacacagaatatccagttatggataaaaatgagctggtgcagaaggccaagctggccgagcaggcagagcgatacgatgacatggcagcctgcatgaagtctgtcactgagcaaggagccgagctgtctaacgaggagaggaaccttctctctgttgcttataaaaacgttgtaggagcccgtaggtcatcttggagggtcgtctcgagtattgagcagaagacggaaggtgctgagaaaaagcagcagatggctcgagaatacagagagaagatcgagatggagctgagggacatctgcaacgacgtactgtctcttttggaaaagttcttgatccccaatgcttcgcagccagaaagcaaagtcttctatttgaaaatgaagggtgactactaccgctacttggctgaggttgctgctggtgatgacaagaaaggaattgtggaccagtcacagcaagcataccaagaagcatttgaaatcagcaaaaaggagatgcagccgacacaccccatcagactgggtctggccctcaacttctctgtgttctactatgagatcctgaactccccagagaaagcctgctctcttgcaaaaacagcttttgatgaagccattgctgaacttgatacattaagtgaagagtcgtacaaagacagcacgctaataatgcagttactgagagacaacttgacattgtggacatcggatacccaaggagacgaagcagaagcgggagaaggaggggaaaattaaccggccttccaacctttgtctgcctcattctaaaatttacacagtagaccatttgtcatccatgctgtcccacagatagtttttttgtttacgatttatgacaggtttatgttacttctatttgaatttctatatttcccatgtggttttatgttttaatattaggggagtagagccagttaactttagggagttactcgttttcatcttgaggtggccaatatgggatgtggaatttttacatgagttacacatgtttggcatagttagtacttttggtccattgtggcttcagaagggccagtgttcaaactgcttccatgtctaagcaaagaaaactgcctacatattggtgtgtgctggcggggaataaacaggataatgggtccagtcatgagtgtagtctgtgtgggtactgtaaggcttggagcacttgtgaggctgggacacgaacaccctgtggatgcacgctaagaccgtgtgtctgcgtgcacacccttgaccacagctccagaagttgtctgtagacaaagttgtgacccaatttactctgataagggcagaaacggttcacattccattatttgtaaagttacctgctgtttgctttcattatttttgctacacattttatttgtatttaaatgttttaggcaatctaagaacaaatgtaaaagtaaagatgcagtacaaacgagttgcttggtgttcccggctccatgcggatcaagcacagcggtaaacaaaatcccatgtatttaactttttttttttaagttttttgttttgttttgtttttgcttttgtgattttttttctctttttgatacttgcctaacatgcatgtgctgtaaaaatagttaacagggaaataacttgagatgacg

SEQ ID NO: 33 - protein, 246 aa, Rattus norvegicus
  Protein 14-3-3-protein eta subtype
MGDREQLLQRARLAEQAERYDDMASAMKAVTELNEPLSNEDRNLLSVAYKNVVGARRSSWRVISSIEQKTMADGNEKKLEKVKAYREKIEKELETVCNDVLALLDKFLIKNCNDFQYESKVFYLKMKGDYYRYLAEVASGEKKNSVVEASEAAYKEAFEISKEHMQPTHPIRLGLALNFSVFYYEIQNAPEQACLLAKQAFDDAIAELDTLNEDSYKDSTLIMQLLRDNLTLWTSDQQDEEAGEGN

SEQ ID NO: 34 - DNA, 1689 bp, Rattus norvegicus
  cDNA 14-3-3-protein eta subtype
tgcagccagctagcgagaaggcgcgagcggcggcgcagccagcagcctcccgccagccggcgagccagtgcgcgtgcgcggcggcggcctcggcggcgaccgggaagcggacgggcgggcgaggcgagcgaggcaggcggtgcgggcgtgcgaggcgaggccgatcgcgagcgacatgggggaccgagagcagctgctgcagcgggcgcgactggcggagcaggcggagcgctacgacgacatggcctccgccatgaaggcggtgacagagctgaatgaacctctatctaatgaagatagaaatctcctctctgtggcctacaagaatgtagttggtgccaggcgatcttcttggagggttattagtagcattgagcagaaaaccatggcagatgggaatgagaagaagctggagaaagtcaaagcctatcgggagaagattgagaaggagctggagacagtttgcaatgatgtcttggctctgctcgacaagttccttatcaagaactgcaatgattttcagtacgagagcaaggtgttctacctgaaaatgaagggcgattactaccgctacctggcagaggtggcttctggggagaagaaaaacagtgtggttgaagcttctgaggcagcgtataaggaagccttcgaaatcagcaaagagcacatgcagccaacacaccccatccggcttggcctggccctcaatttttctgtgttctactatgagatccagaatgcaccagagcaggcctgcctcttagccaaacaagccttcgatgatgctatagctgagctggacacattaaacgaggattcctataaggactccactctcatcatgcagttgctgcgagacaacctcaccctctggacgagcgaccagcaggatgaagaagccggagaaggcaactgaagacccatcaggtccctggcccttcctttacccaccacccccattatcactgattcttccttgccacaatcactatatctagtgctaaacctatctgtattggcagcacagctattcagatctgccctcctgtcccttggaagcagtttcagataaaccttcatgggcatttgctggactgatggttgctttgagccacagagcgctccctttttgaattgtgcagagaagtgtgttctgaacgaggcattttattatgtctgttgatctgtagcaaatccatgtgatggtaattgagtgtagaaaggagaattagccaacacaggctatggctgctatttaaaacaagctgatagtgtgttgttaagcagtacatctcgtgcatgcaaaaatgaatttgaccctctcaccccttctttcagctaatggaaactgacacacgacaacttgttccttcaccatcagctttataaactgtttctcgtgagctttcaggcccctgctgtgcctctttaaattatgatgtgcgcacaccttcttttcaatgcaatgcatcagaggtttttgatatgtgtaacttttttttttggttgtgattaagaatcatggatttattttttgtaactctttggctattgttcttgtgtaccctgacagcatcatgtgtgtcaacctgtgtcaatcatgatgggtggttatgaaatgccagattgctaaaataaatgttttggacttaaaaagagtaaataaatgctgctttggggatattaaaa